(19) United States
(12) Patent Application Publication
(10) Pub. No.: US 2004/0023221 A1
(43) Pub. Date: Feb. 5, 2004

(54) Method of designing and selecting polynucleotide sequences

(76) Inventors: Petr Pancoska, Evanston, IL (US); Vita Janota, Praha (CS); Albert S. Benight, Schaumburg, IL (US); Richard S. Bullock, Chicago, IL (US); Peter V. Riccelli, Tinley Park, IL (US)

Correspondence Address: BLAKE, CASSELS & GRAYDON LLP, BOX 25, COMMERCE COURT WEST, 199 BAY STREET, SUITE 2800, TORONTO, ON, M5L 1A9, CA

(21) Appl. No.: 10/203,383

(22) PCT Filed: Feb. 8, 2001
(86) PCT No.: PCT/CA01/00141

(51) Int. Cl. C12Q 1/68; G06F 19/00; G01N 33/48; G01N 33/50

(52) U.S. Cl. 435/6; 702/20

(57) ABSTRACT

Methods for processing sequences so as to be capable of selecting a family of polynucleotide sequences in which any two sequences with in the family meet predetermined criteria, particularly with respect to the degree of homology between the sequences.

### FIELD OF THE INVENTION

The invention relates generally to the area of bioinformatics and in particular to methods for processing sequences so as to be capable of selecting a family of polynucleotide sequences in which any two sequences within the family meet predetermined criteria, particularly with respect to the degree of homology between the sequences.

### BACKGROUND OF THE INVENTION

Specific hybridization of oligonucleotides and their analogs is a fundamental process that is employed in a wide variety of research, medical, and industrial applications, including the identification of disease-related polynucleotides in diagnostic assays, screening for clones of novel target polynucleotides, identification of specific polynucleotides in blots of mixtures of polynucleotides, therapeutic blocking of inappropriately expressed genes and DNA sequencing.

In large part, the success of hybridization using oligonucleotides depends on minimizing the number of false positives and false negatives. Such problems have made the simultaneous use of multiple hybridization probes in a single experiment, particularly in the analysis of multiple gene sequences on a gene chip array very difficult. For example, in certain binding assays, a family of nucleic acid molecules is bound to a chip with the desire that a given “target” sequence will bind selectively to its complement attached to the chip. In one such assay, known as the “zip chip”, a family of nucleic acid molecules, the “addresses”, each different from each other are set out on a grid. One way of building the nucleic acid molecules of the “addresses” is by a stepwise addition of tetramers, which are laid down as rows followed by columns of individual tetramers on the chip. Once the rows are laid down, the chip is rotated 90° and the columns are then laid down. Each row and column is separated by a space, which results in a grid of full-length nucleic acid molecules separated by 12mers in the spaces between the full length tags. The end result of this method of generating polynucleotide sequences of 24 nucleotides in length (24mer) from a set of four possible tetramers is that each 24 mer “address” differs from its nearest 24mer neighbour by 3 tetramers. Further, if each tetramer differs from each other by at least two nucleotides, then each 24mer will differ from the next by at least six nucleotides. A unique “zip code” sequence is ligated to a label in a target dependent manner, resulting in a unique “zip code” which is then allowed to hybridize to its address on the chip. To minimize cross-hybridization of a “zip code” to other “addresses”, the hybridization reaction is carried out at temperatures of 75-80° C. Due to the high temperature conditions for hybridization, neighbouring 24mers do not hybridize to any complementary sequences and represent ‘dead zones’.

Thus, while it is desirable with such a chip to have, at once, a large number of address molecules (several thousand per chip), the address molecules should each be highly selective for its own complement zipcode. While such a chip provides the advantage that the family of molecules making up the grid is entirely of design, and does not rely on sequences as they occur in nature, the provision of a family of molecules, which is sufficiently large and where each individual member is sufficiently selective for its complement over all the other zipcode molecules (i.e., where there is sufficiently low cross-hybridization, or cross-talk) continues to elude researchers. It is in this area that the invention described herein is of particular usefulness.

### Definitions

An important aspect of this invention is a method for creating a family of block sequences which family is in turn useful for creating a family of nucleic acid molecules having a selected property.

In the context of this invention, a “block sequence” is a symbolic representation of a sequence of blocks. In its most general form a block sequence is a representative sequence in which no particular value, mathematical variable, or other designation is assigned to each block of the sequence.

In the context of this invention, each block of a block sequence can be assigned a particular or specific designation so as to show its relationship to other blocks. Thus, for example, one block may be assigned the designation “1” and another block may be assigned the designation “2”. Each of these two blocks thus has a “specific fixed designation”. In this particular example, because “1” and “2” are different from each other, each specific designation is different (i.e., has a different value, represents a different topological position, represents a different tetrameric sequence, etc.) from the other. In the preferred embodiment of this invention, specific fixed designations are simply the numerals “1”, “2”, “3”, etc. Ultimately, according to this invention, a specific fixed designation can correspond to a particular nucleic acid sequence, or to a single nucleic acid. As will be seen, such designations are assigned to blocks of sequences as part of the method of this invention in obtaining the desired family of nucleic acid molecules.

In the context of this invention, “topological sequences” are created. A “topological sequence” notionally represents a family of block sequences wherein each member of the family is composed of blocks having a specific designation. As described in connection with preferred embodiments, topological sequences are used to develop criteria for generating sequence templates (see below) which are in turn used to generate specific block sequences. Each block position of a topological sequence is assigned, either an “arbitrary fixed designation”, Φ, or a “variable designation”, μ. A block occupying a position assigned an arbitrary fixed designation, is alternatively referred to herein as a “core” position or block of a sequence. When such a designation is assigned to a position of a topological sequence, it is referred the “topology” assigned to that position. Thus, one possible topological sequence is μ₁-μ₂-Φ₁-Φ₂-Φ₃-Φ₄. This topological sequence represents a family of block sequences each of which is six blocks in length. Within the family of sequences notionally represented by this topological sequence, the third, fourth, fifth and sixth positions, the “Φ” positions, can be assigned any designation. The designations which can be assigned to the first and second positions, the “μ” positions, are restricted and depend upon designations already assigned to one or more of the “Φ” positions. Thus, for example, it may be that μ₁ is not permitted to be the same as Φ₄, i.e., μ₁≠Φ₄. It is also possible for restrictions to apply between variable designations of a given topological sequence, for example μ₁≠μ₂. It is also possible for restrictions to apply between different topological sequences. For example, according to a particular embodiment, the topological sequences μ₁-μ₂-Φ₁-Φ₂-Φ₃-Φ₄ and μ₃-Φ₁-μ₄-Φ₂-Φ₃-Φ₄ are created and μ₁≠μ₂, μ₁≠μ₃, μ₂≠Φ₁, μ₂≠Φ₄, μ₃≠μ₄.

“Sequence templates” are created according to this invention. A sequence template, defined in accordance with criteria developed using the topological sequences, is used as a basis for the generation of a family of block sequences in which each block is assigned a specific fixed designation according to a preferred aspect of this invention. A template for generating a family of block sequences wherein each member of the family has six blocks thus contains six “template blocks”. Associated with each template block of a sequence template is one or more rules for determining the specific fixed designations that can be assigned to a block in that position of a particular block sequence.

As demonstrated below in connection with a preferred embodiment block sequences, in which each block is assigned a specific designation, generated using sequence templates are graphed to obtain families of sequences wherein any two members of a family satisfy defined criteria between themselves. A simple graph G is a pair (V, E) where V represents the set of vertices of the simple graph and E is a set of un-oriented edges of the simple graph. An edge is defined as a 2-component combination of members of the set of vertices. In other words, in a simple graph G there are some pairs of vertices that are connected by an edge. In our application a graph is based on nucleic acid sequences generated using sequence templates and vertices represent DNA sequences and edges represent a relative property of any pair of sequences.

The term “incidence matrix” as used herein is a well-defined term in the field of Discrete Mathematics. The incidence matrix is a mathematical object that allows one to describe any given graph. For the subset of simple graphs used herein, the simple graph G=(V,E), and for a pre-selected and fixed ordering of vertices, V={v₁,v₂, . . . ,vₙ}, elements of the incidence matrix A(G)=[aᵢⱼ] are defined by the following rules:

(1) aᵢⱼ=1 for any pair of vertices {vᵢ,vⱼ} that is a member of the set of edges; and

(2) aᵢⱼ=0 for any pair of vertices {vᵢ,vⱼ} that is not a member of the set of edges.

This is an exact unequivocal definition of the incidence matrix. In effect, one selects the indices: 1, 2, . . . n of the vertices and then forms an (n×n) square matrix with elements aᵢⱼ=1 if the vertices vᵢ and vⱼ are connected by an edge and aᵢⱼ=0 if the vertices vᵢ and vⱼ are not connected by an edge.

To define the term “class property” as used herein, the term “complete simple graph” or “clique” must first be defined. The complete simple graph is required because all sequences that result from the method described herein should collectively share the relative property of any pair of sequences defining an edge of graph G, for example not violating the threshold rule that is, do not have a “maximum simple homology” greater than a predetermined amount, whatever pair of the sequences are chosen from the final set. It is possible that additional “local” rules, based on known or empirically determined behavior of particular nucleotides, or nucleotide sequences, are applied to sequence pairs in addition to the basic threshold rule.

In the language of a simple graph, G=(V, E), this means in the final graph there should be no pair of vertices (no sequence pair) not connected by an edge (because an edge means that the sequences represented by vᵢ and vⱼ do not violate the threshold rule).

Because the incidence matrix of any simple graph can be generated by the above definition of its elements, the consequence of defining a simple complete graph is that the corresponding incidence matrix for a simple complete graph will have all off-diagonal elements equal to 1 and all diagonal elements equal to 0. This is because if one aligns a sequence with itself, the threshold rule is of course violated, and all other sequences are connected by an edge.

For any simple graph, there might be a complete subgraph. First, the definition of a subgraph of a graph is as follows. The subgraph Gs=(Vs,Es) of a simple graph G=(V,E) is a simple graph that contains the subsets of vertices Vs of the set V of vertices and inclusion of the set Vs into the set V is immersion (a mathematical term). This means that one generates a subgraph Gs=(Vs,Es) of a simple graph G in two steps. First select some vertices Vs from G. Then select those edges Es from G that connect the chosen vertices and do not select edges that involve non-selected vertices.

We desire a subgraph of G that is a complete simple graph. By using this property of the complete simple graph generated from the simple graph G of all sequences generated by the template based algorithm, the pairwise property of any pair of the sequences (violating/non-violating the threshold rule) is converted into the property of all members of the set, termed “the class property”.

By selecting a subgraph of a simple graph G that is a complete simple graph, this assures that, up to the tests involving the local rules described herein, there are no pairs of sequences in the resulting set that violate the threshold rule, also described above, independent of which pair of sequences in the set are chosen. This feature is called the “desired class property”.

In a first broad aspect, the invention is a method of processing a family of topological block sequences useful in creating a family of nucleic acid sequences. Families of nucleic acid molecules having the derived sequences can be synthesized. The method includes steps of:

(a) providing first and second topological block sequences, each sequence having a predetermined number of core blocks and a predetermined number of variable blocks;

(b) aligning the first and second sequences with each other such that at least one block of the first sequence is paired with at least one block of the second sequence in an aligned arrangement;

(c) assigning conditions to the variable blocks of the first and second sequences as necessary to provide that, in the arrangement of (b), the sum of (i) the number of pairs of aligned core blocks, and (ii) the number of pairs of aligned variable blocks, in which both variable blocks are permitted to have the same designation, does not exceed a predetermined threshold; and

(d) storing the conditions assigned in (c) for each variable block of the first and second sequences in a computer readable medium in association with the respective first and second sequences.

Step (a) can include providing first and second topological sequences which have the same topology as each other, and the first and second sequences can be aligned with each other such that each core block of one sequence is paired with a core block of the other sequence. Alternatively, or additionally, step (a) includes providing first and second topological sequences having topologies different one from the other, and the first and second topological sequences are aligned with each other such that the number of pairs of aligned core blocks is maximized.

The method can also include steps of:

(e) providing a database of specific block sequences;

(f) determining which of the plurality of specific block sequences meet the conditions assigned in step (c); and

(g) storing the specific block sequences determined in step (f) to meet the conditions assigned in step (c) into a database.

The topological sequence can be any length which can ultimately serve as a basis for obtaining nucleic acid sequences of the desired length. Preferably, each topological block sequence has at least five blocks and at least three of the blocks are core blocks. In the preferred embodiment described below, each topological sequence has six blocks.

The method often includes a step (h) of repeating steps (b) through (d) for a different said aligned arrangement of pairs of topological block sequences, having topologies different one from the other, of step (b). Alternatively, or additionally, the method can include step (i) of repeating steps (b) through (d) for a different pair of first and second topological block sequences which can have the same topology as each other.

In particular embodiments, first and second of said core blocks of each topological sequence of the family are each located adjacent a variable block; and the method further includes step (j) of, prior to step (c), assigning to the first and second core blocks, the condition that the first and second core blocks each have the same designation.

It is also possible for first and second core blocks of each topological sequence of the family to be each located adjacent a variable block; and for the method to include step (j) of, prior to step (c), assigning to the first and second core blocks, the condition that the first core block has a different designation from the second core block.

Preferably, at least one variable block of each topological sequence of the family is located in a terminal position of a said topological sequence.

As described above, the number of sequences in a family of sequences of a given length (L) and derived using a fixed number of specific designations (K) is fixed. This is true whether one is speaking of block sequences or nucleotide sequences and whether K represents a fixed number of specific designations (as notionally used for block sequences) or a fixed number of xmers used in deriving a family of nucleotide sequences. Thus, if L=6 and K=4, the number of sequences in the family 4⁶=4096. A primary object of the present invention is to eliminate from such a family of sequences a number of the sequences so that when sequence-to-sequence comparisons of pairs of remaining individual sequences are made to determine whether such pairs share a given property, (e.g., share no more than a given maximum amount of simple homology with each other), the number of comparisons required to be made is reduced. Any combination of the steps provided according to the methods disclosed herein that obtains such a subgrouping of a family of sequences finds utility in reducing the number of comparisons required to be made to obtain a family of nucleotide sequences having the desired property.

In a second embodiment, the invention is a method of processing a family of topological block sequences useful in creating a family of nucleic acid molecules having a desired sequence property in which the method includes:

(a) providing a first pair of first and second topological block sequences, each sequence having c core blocks and v variable blocks, c and v being natural numbers, the first and second topological sequences each having a first topology;

(b) aligning the first and second sequences with each other such that each core block of one sequence is paired with a core block of the other sequence and each variable block of one sequence is paired with a variable block of the other sequence;

(c) assigning conditions to the variable blocks of the first and second sequences that are necessary to provide that the sum of (i) the number of pairs of aligned core blocks, and (ii) the number of pairs of aligned variable blocks, in which both variable blocks are permitted to have the same designation, does not exceed a predetermined threshold; and

(d) storing the conditions determined for each variable block of the first and second sequences in a computer readable medium in association with the respective first and second sequences.

This second embodiment can further include providing a second pair of first and second topological block sequences, each sequence having c core blocks and v variable blocks, the topological sequences of the second pair each having a second topology, and repeating steps (b) through (d) for the second pair of first and second topological block sequences. The method can include:

(e) providing a database of specific block sequences;

(f) determining which of the plurality of specific block sequences meet the conditions assigned in step (c); and

(g) storing the specific block sequences determined in step (f) to meet the conditions assigned in step (c) into a database.

The second embodiment method can include:

(1) providing a third pair of first and second topological block sequences, each sequence having c core blocks and v variable blocks, wherein the topological sequences have different topologies one from the other and wherein the topology of one said sequence is the same as the topology of one of the first and second pairs of topological sequences and wherein the topology of the other said sequence is the same as the topology of the other for the first and second pairs of topological sequences;

(2) aligning the first and second topological block sequences provided in step (1) with each other such that the number core blocks in paired alignment with each other is maximized; and

(3) assigning conditions to the variable blocks of the first and second sequences of step (2) that are necessary to provide that the sum of (1) the number of pairs of aligned core blocks, and (2) the number of pairs of aligned variable blocks, in which both variable blocks are permitted to have the same designation, does not exceed the predetermined threshold; and

(4) storing the conditions determined for each variable block of the first and second sequences in a computer readable medium in association with first and second sequence templates, respectively, corresponding to the respective first and second topological sequences.

Preferably, the sum of c and v is at least five, or six and each of v and c is at least two. Preferably, v is two.

Again, at least one variable block of each topological sequence can be located in a terminal position of the topological sequence.

The method can include the further step of, prior to step (3), assigning to the first and second core blocks, the condition that the first and second core blocks each have the same designation.

The second embodiment method can include, for each specific block sequence stored in step (g), determining whether the block sequence meets the conditions stored in step (4) of in associate with a first said sequence template; and storing said sequences into a database. Additionally, there can be a step of determining the maximum number of specific block sequences that meet the conditions stored in step (4) in association with the first said sequence template.

The second embodiment method can include, for each specific block sequence stored in step (g), determining whether the block sequence meets the conditions stored in step (4) in association with a second said sequence template; and storing said sequences into a database. Again, the method can include the step of, prior to step (3), assigning to the first and second core blocks, the condition that the first and second core blocks have different designations, one from the other.

The method can further include the steps of (h) selecting a first sequence from the database of step (g); (i) selecting a second sequence from the database of step (g); (j) aligning the first and second sequences so as to maximize the number of paired blocks having the same designation; (k) determining the number of matching pairs; (l) arranging the first and second sequences of step (j) in a matrix, wherein: (l)(i) if the number of paired blocks having the same designation is less than or equal to the threshold of step (c), then the first and second sequences are associated with each other in the matrix; and (l)(ii) if the number of paired blocks having the same designation is greater than the threshold of step (c), then the first and second sequences are non-associated with each other in the matrix; and (m) repeating steps (h) to (l) for a different pair of first and second sequences so as to form one or more cliques or groups of sequences, each clique (group) comprising a set of sequences wherein each sequence is associated with every other sequence.

Of course, a person skilled in the art understands that the object of step (m) is to obtain a grouping of sequences, the family of which has a preselected property. Any suitable mathematical operation which accomplishes the object can be used.

The method can further include, for a said clique or grouping: (A) assigning a nucleotide or an x-mer to each specific designation to obtain a nucleic acid sequence corresponding to each sequence of said clique: (B) selecting first and second of the nucleic acid sequences of step (A); (C) aligning the first and second sequences so as to maximize the number of paired matching nucleotides; (D) determining the number of matching nucleotides; (E) arranging the first and second sequences of step (B) in a matrix, wherein: (F)(i) if the number of pairs of matching nucleotides is less than or equal to a predetermined threshold, then the first and second sequences are associated with each other in the matrix; and (F)(ii) if the number of pairs of matching nucleotides is greater than the threshold, then the first and second sequences are non-associated with each other in the matrix; and (G) repeating steps (B) to (F) for a different pair of first and second sequences so as to form one or more cliques, each clique comprising a set of sequences wherein each sequence is associated with every other sequence.

In a preferred method, each block sequence is six blocks in length, and each x-mer is a 4-mer.

In another embodiment, the invention is a method of processing block sequences, the method comprising;

(I) providing a database comprising a plurality specific block sequences six blocks in length;

(II) determining which of the plurality of block sequences meet the conditions assigned in step (c) of claim 138 for a predetermined threshold for a first toplogical sequence six blocks in length;

(III) storing the specific block sequences determined in step (II) to meet the assigned conditions into a database;

(IV) repeating steps (II) and (III) for a second topological sequence six blocks in length;

(V) determining whether each specific block sequence stored in step (III) meet conditions assigned according to step (iii) of claim 141 wherein the first and second topological block sequences of step (iii) correspond to the first and second toplogical sequences of steps (II) and (IV);

(VI) storing the specific block sequences determined in step (V) to meet the assigned conditions into a database;

(VII) selecting first and second sequences from the database of step (VI);

(VIII) aligning the first and second sequences of step (VII) so as to maximize the number of paired blocks having the same designation;

(IX) determining the number of matching pairs of blocks of step (VIII);

(X) storing matched pair blocks onto a computer readable medium in association with each other, as in a matrix, wherein: (X)(i) if the number of paired blocks having the same designation is less than or equal to the threshold, then the first and second sequences are associated with each other; and

(XI) repeating steps (VIII) to (X) for a different pair of first and second sequences so as to form one or more cliques, each clique comprising a set of sequences wherein each sequence is associated with every other sequence.

In a fourth broad aspect, the invention is a method of processing a family of topological block sequences useful in creating a family of nucleic acid molecules, the method comprising:

(a) providing first and second topological block sequences, each sequence having a predetermined number of core blocks and a predetermined number of variable blocks;

(b) aligning the first and second sequences with each other such that at least one block of the first sequence is paired with at least one block of the second sequence in an aligned arrangement;

(c) assigning conditions to the variable blocks of the first and second sequences, as necessary, such that the sum of (i) the number of pairs of aligned core blocks, and (ii) the number of pairs of aligned variable blocks, in which both variable blocks are permitted to have the same designation, does not exceed a predetermined threshold;

(d) storing the conditions determined for each variable block of the first and second sequences in a computer readable medium in association with the respective first and second sequences;

(e) optionally, repeating steps (b) through (d) for a different said aligned arrangement of step (b); and

(f) optionally, repeating steps (b) through (e) for a different pair of first and second topological block sequences.

This embodiment can include:

(h) providing a database of specific block sequences, each block of each sequence having a specific designation associated therewith;

(i) determining which of the plurality of specific block sequences meet the conditions assigned in step (c);

(j) storing the specific block sequences determined in step (i) to meet the conditions assigned in step (c) into a database.

The method can include assigning an x-mer to each specific designation of a block sequence of step (j). The first and second sequences of step (b) can have the same topology as each other. The first and second sequences of step (b) can have a different topology from each other. Again, each topological block sequence preferably has at least 5 blocks, but there can be 6 blocks, 7 blocks, or 8 or more blocks. In the disclosed embodiment, used to obtain families of 24mer nucleic acid sequences, each topological block sequence consists of 6 blocks.

The number of core blocks can exceed the number of variable blocks. In the disclosed embodiment, the number of core blocks is 4 and the number of variable blocks is 2 and at least one variable block is a terminal block of each topological block sequence.

The method can thus be conducted such that:

each topological sequence has 4 core blocks 2 variable blocks;

the first and second sequences of step (b) have the same topology as each other; and

at least one variable block of each topological block sequence is a terminal block.

In a fifth embodiment, the invention is a method of processing a family of topological block sequences useful in creating a family of nucleic acid molecules, the method comprising:

(a) providing a first pair of first and second topological block sequences, each sequence having c core blocks and v variable blocks, c and v being natural numbers, the first and second topological sequences having the same topology as each other;

(b) aligning the sequences with each other such that the core blocks of each sequence are paired with each other and the variable blocks of each sequence are paired with each other in an aligned arrangement;

(c) assigning conditions to the variable blocks of the first and second sequences that are necessary to preclude the sum of (i) the number of pairs of aligned core blocks, and (ii) the number of pairs of aligned variable blocks, in which both variable blocks are permitted to have the same designation, from exceeding a predetermined threshold; and

(d) storing the necessary conditions determined for each variable block of the first and second sequences in a computer readable medium in association with the respective first and second sequences.

This method can include step (e) of providing a second pair of said first and second topological block sequences, each sequence having c core blocks and v variable blocks, wherein the topology of the second pair of sequences is different from the topology of the first pair of sequences, and conducting steps (b) to (d) for the second pair of sequences.

The method can include step (f) of providing a database of specific block sequences, each block of each sequence having a specific designation associated therewith, (g) determining which of the plurality of specific block sequences meet the conditions stored in step (d) in association with the first pair of topological sequences; (h) repeating step (g) for the conditions stored in step (d) in association with the second pair of topological sequences; and (i) storing the specific block sequences determined in steps (g) and (h) into a database.

The can include the steps of (j) selecting a first sequence from the database of step (i); (k) selecting a second sequence from the database of step (i); (l) aligning the first and second sequences so as to maximize the number of paired blocks having the same designation; (m) determining the number of matching pairs; (n) arranging the first and second sequences of step (l) in a matrix, wherein: (n)(i) if the number of paired blocks having the same designation is less than or equal to the threshold of step (c), then the first and second sequences are associated with each other in the matrix; and (n)(ii) if the number of paired blocks having the same designation is greater than the threshold of step (c), then the first and second sequences are non-associated with each other in the matrix; and (o) repeating steps (j) to (n) for a different pair of first and second sequences of step (i).

In a final sixth broad aspect, the invention includes a method of processing a family of topological block sequences useful in creating a family of nucleic acid molecules, the method comprising:

(a) providing a family of topological block sequences, each sequence of the family having a predetermined first number of core blocks and a predetermined second number of variable blocks;

(b) selecting first and second sequences of the family;

(c) aligning the first and second sequences with each other such that at least one block of the first sequence is paired with at least one block of the second sequence in an aligned arrangement;

(d) determining conditions assignable to the variable blocks of the first and second sequences, as necessary, to maintain the condition that the sum of (i) the number of pairs of aligned core blocks, and (ii) the number of pairs of aligned variable blocks, in which both variable blocks are permitted to have the same designation, does not exceed a predetermined threshold; and

(e) storing the conditions determined for each variable block of the first and second sequences in a computer readable medium in association with the respective first and second sequences;

(f) optionally, repeating steps (c) through (e) for a different arrangement of step (c); and

(g) optionally, repeating steps (b) through (f) for different first and second toplogical sequences.

### BRIEF DESCRIPTION OF THE DRAWINGS

FIGS. 1A, 1B and 1C summarize the method of designing and selecting polynucleotide sequences with a desired property.

FIG. 2 shows all fifteen possible arrangements (topological sequences) of placing two variable block elements at all six positions p-p₆ of a topological sequence.

FIG. 3 shows an example of topological arrangements of the Φ block elements to force pairwise alignments characterized by the threshold value of ˜66% for a 24mer made up of 6 blocks of 4mers.

FIG. 4 shows the two subsets of Φ block elements that will generate well-behaved subsets of polynucleotide sequences i.e., type ii Φ block elements and type ij Φ block elements.

FIG. 5 shows four sequence templates and demonstrates the sequence-generating algorithm.

FIG. 6 shows the sliding rule for pairwise comparison of sequences generated from the sequence templates to check for good alignment.

FIG. 7 shows the incidence matrix constructed of rows of sequence i and columns of sequence j and the representation of the incidence matrix as a simple graph.

FIG. 8 shows the application of the local rules to a set of sequences generated from different complete subgraphs or “cliques” of the simple graph defined by the incidence matrix of FIG. 7.

### DETAILED DESCRIPTION OF THE INVENTION

Conceptually, the method of generating a maximum number of minimally cross-hybridizing polynucleotide sequences as, described herein, can be summarized as follows. First, a set of topological sequences of a given length are created based on a given number of block elements. Thus, if a family of polynucleotide sequences 24 nucleotides (24 mer) in length is desired from a set of 6 block elements, each element comprising 4 nucleotides, then a family of 24 mers is generated considering all positions of the 6 block elements. In this case, there will be 6⁶ ways of assembling the 6 block elements to generate all possible polynucleotide sequences 24 nucleotides in length. Constraints are now imposed on the topological sequences to force pairwise alignments characterized by the number of block elements which can be allowed to be identical between any two pairs of polynucleotide sequences. This number can be of any value depending on the degree of simple homology desired between any two polynucleotide sequences. Thus, in the case where about 66% simple homology is desired, four out of six of the blocks can be identical between any two pairs of topological sequences. The four identical blocks are the “core” blocks and the remaining two blocks are the variable blocks. The constraints are expressed as a set of rules on the identities of the blocks which can be placed in the two variable positions such that when any two of the six block elements are placed in the variable positions, the percentage homology between any two topological sequences will not exceed the degree of simple homology desired between these two sequences. All polynucleotide sequences generated for a certain topological sequence which obey the rules are output to a database. Each topological sequence will generate a database of polynucleotide sequences which sequences within a database will obey the rules but between databases will not necessarily obey the rules. If the number of sequences desired at this point within a database is not large enough, the sequences of databases are combined. Keeping in mind that a subset of these combined sequences can still exceed the desired 66% simple homology (since the rules apply to sequences within a database and not between databases), a new set of rules are created which include placing constraints on the identities of the two boundary positions of the four core blocks for a given template. By doing so, two subsets of sequence templates, one with boundary positions having identical block elements and another having boundary positions with different block elements, will each generate a database of polynucleotide sequences which sequences within a database will obey the rules imposed and not exceed the 66% simple homology rule between any two polynucleotide sequences in that database. At this point constraints have been placed on all but two of the six block elements of a given template. Templates are selected such that these are the two block elements which are adjacent to each other and which are located in the center of the core block. By filling these two positions with any of the six blocks, some of the sequences can still exceed the 66% simple homology between any two sequences. An incidence matrix is next constructed of rows of sequences with identical block elements in the boundary positions of the core blocks and columns of sequences with different block elements in the boundary positions in the following manner. Each sequence is compared with every other sequence and sequences which exceed the 66% threshold are assigned an incidence matrix element of 0. Those that do not are assigned an incidence matrix element of 1. The incidence matrix is stored in a database. The incidence matrix can be thought of as a simple graph and the sequences with the desired property of being minimally cross hybridizing as a clique of the simple graph, which may have multiple cliques. While sequences within each clique meet the threshold, this may not be so for sequences between cliques. For the set of sequences from each clique, local rules i.e., comparison of each sequence with every other sequence at the nucleotide level by moving each sequence with respect to the other one nucleotide at a time and counting the number of common nucleotides, are applied. Again, these comparisons provide an incidence matrix where an element of 0 is assigned to each pair exceeding the threshold of common nucleotides (16 in the example), and an element of 1 is assigned otherwise. The incidence matrix represents a graph, where vertices correspond to sequences, in which cliques are selected. The resulting sequences are tabulated and if their number is sufficient, the method is complete. However, if the number of sequences is still smaller than desired, then additional block elements different from the original six are chosen and the entire process repeated until the desired number of sequences are generated once a clique containing a suitably large number of sequences is found, the sequences are tested to determine if it is possible to obtain a set of minimally cross-hybridizing sequences therefrom.

A preferred method of generating a family of nucleotide sequences is now described in detail. Here, the length of the polynucleotide, N, is 24 and the polynucleotide is assembled from 6 blocks wherein each block is selected from a family of 6 block elements and each block element represents a nucleotide sequence 4 nucleotides in length. The number of block elements in the family of block elements is more generally referred to as K, and so here, K=6. The number of nucleotides in a block element is generally referred to as b. The number of blocks in a sequence (i.e., the length of the sequence measured in the number blocks assembled to obtain the block sequence) is 6. The number of blocks in a sequence is more generally referred to as L, so here L=6.

In this example, b=4 and K=6, so the total possible number of polynucleotides obtainable is 6⁶=46,656. That is, there are 6 (K) possible ways of filling the first position of the sequence, 6 (K) possible ways of filling the second position of the sequence, etc. for a total of 6 (L) positions. Thus, K^{L}=46,656. Similarly, if b=3 and K=9, then the total number of ways to assemble a set of nine trimers to build a set of polynucleotide sequences whose length N=27 (27mer) is K⁹=387,420,489.

Within the complete set of all possible numbers of Nmer sequences (equivalently referred to herein as xmers) that can be generated from a set of K bmers, is a subset of sequences with defined characteristics. What is desired, and described in detail herein, is a method of obtaining the largest family, T, wherein the degree of simple homology between any two members of the family is a particular maximum. “Simple homology” between a pair of sequences is defined here as the number of pairs of nucleotides that are matching (are the same as each other) in a comparison of two aligned sequences. “Maximum simple homology” is obtained when two sequences are aligned with each other so as to have the maximum number of paired matching nucleotides.

In the context of hybridization of polynucleotide sequences, it is expected that the complements of two sequences bound to a chip, wherein the bound sequences have a relatively high maximum simple homology (say 95%, or more) would have a greater tendency to cross-hybridize (i.e., bind with the mismatched bound sequence) than if the two bound sequences have a relatively low maximum simple homology (say 66%, or less). It is an object of this invention to provide a method for the generation of a family of nucleotide sequences wherein any two sequences of the family have a maximum degree of simple homology. Put another way, no two nucleotide sequences selected from such a family of sequences have more than a given amount of simple homology with each other, no matter how aligned. (A person skilled in the art understands, of course, because of the complementary nature of sequences that bind with each other, say on a chip, it is sufficient in an analysis of homology to deal with only a single primary set of sequences, say the set bound to the chip, without explicit reference to the complementary set that exists in solution.)

As mentioned above, molecular interactions that are potentially involved in cross-hybridization between two polynucleotide strands are a function of their sequence alignment. Thus, if two or more members of a sequence family (e.g. a family of block sequences) contain a number of identical base pairs that is greater than some critical number of non-identical base pairs greater than the maximum degree of simple homology, cross hybridization is generally expected to occur. To take the present example in which b=4 and K=6 for a family of 24mers, to determine how many sequences do or do not have the critical number of non-identical base pairs, if one were to evaluate all possible alignments one base at a time, between all members of the family to obtain a subset of sequences with a desired property of having a given maximum degree of simple homology, i.e., being minimally cross-hybridizing, the number of comparisons that would have to be made is ½*K^{L}(K^{L}−1)(4b−1)=16,325,517,600 (in the example of 16 base matches).

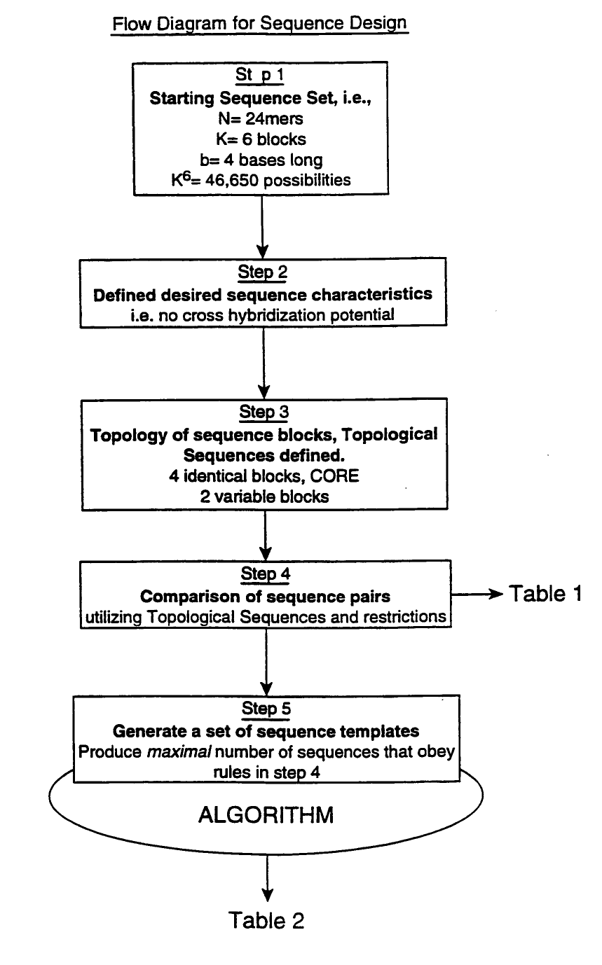

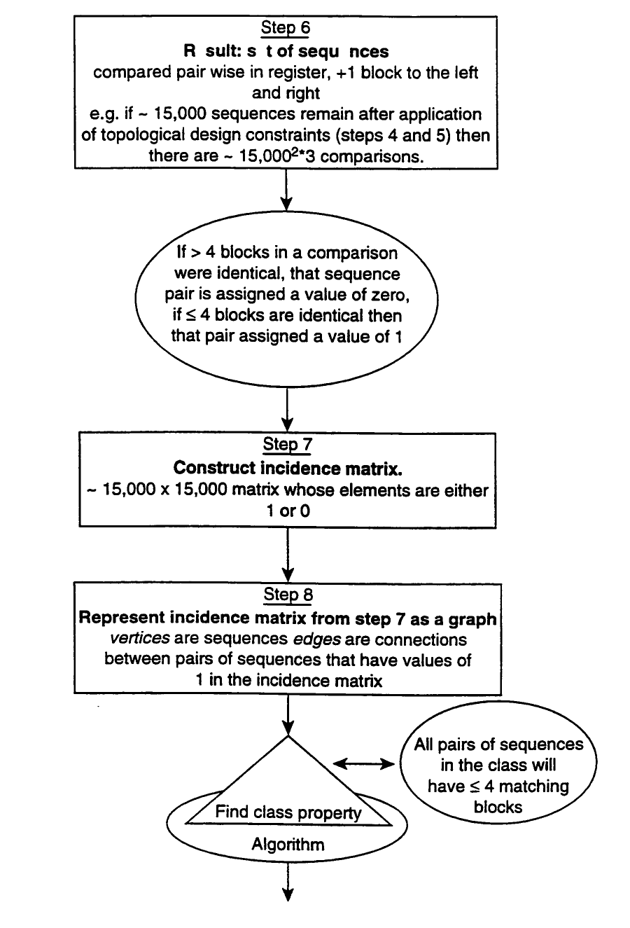

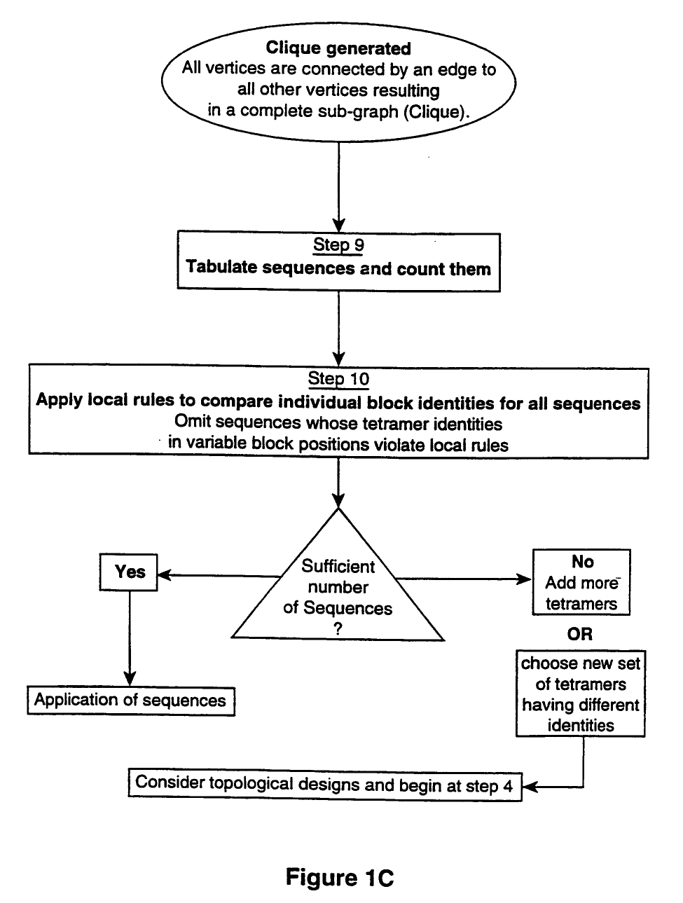

The method described herein, summarized in FIGS. 1A-C as a flow diagram, allows for reducing the comparisons needed to identify a maximum number of minimally cross-hybridizing sequences within the set of all possible sequences. The method leads to the construction and application of a set of topological designs, “sequence templates” which direct assembly of the set of block elements K, each b nucleotides in length to finally generate a family of polynucleotide sequences which are minimally cross-hybridizing. In this particular example, the maximum degree of simple homology that is permitted is 66⅔ percent. This results when 16 out of 24 nucleotides in a pair of sequences (aligned to obtain maximal pairwise matching) match with each other. This approach eliminates the necessity to determine and compare all possible sequence alignments.

Creating Toplogical Sequences

The strategy takes advantage of the requirement that sets of block elements K of bmers can be utilized to assemble the xmers. What is needed is to fill the block sequences with block elements in such a way that the alignment of any pair of sequences is well defined. To achieve this, block positions of a block sequence are divided into two types. The first type are positions called the ‘core’ positions each having an “arbitrary fixed designation”. In pairwise comparisons b-mers in the core positions remain the same. The second type of positions are assigned “variable designations”. The b-mers in the variable positions vary from 1 to K in pairwise comparisons.

Core positions determine the unique alignment of any two polynucleotide sequences. The variable positions are then assigned specific fixed designations to prevent alignment of additional blocks in addition to alignment of the blocks in the core positions. For example, in the generation of a family of xmers where x=24 assembled from a set of bmers where b=4, there are potentially five unique pairwise alignments of bmers in the core positions that can conceivably stabilize unique alignment of any two polynucleotide sequences. These occur when 1, 2, 3, 4 or 5 of the tetramer blocks are identical between any pairs of sequences, corresponding to 16.7, 33.3, 50.0, 66.7, and 83% respectively. Definition of the maximum number of core positions which are allowed to be identical between any pairs of sequences determines how topological sequences are refined to produce set of sequence templates and then applied to generate the maximal set of minimally cross-hybridizing sequences from a set of bmers taken from a set of K block elements.

In the example of a 24mer, where K=6 and b=4, 4 block elements in the core positions and 2 block elements in the variable positions define the limits. For the purpose of sequence design, only this upper limit on the number of blocks in the core and variable regions needs to be considered since all sequences with less than four core positions matching will have the desired property of being minimally cross-hybridizing. That is, sequences with greater than four blocks in common have a greater chance of cross-hybridization than desired according to this example.

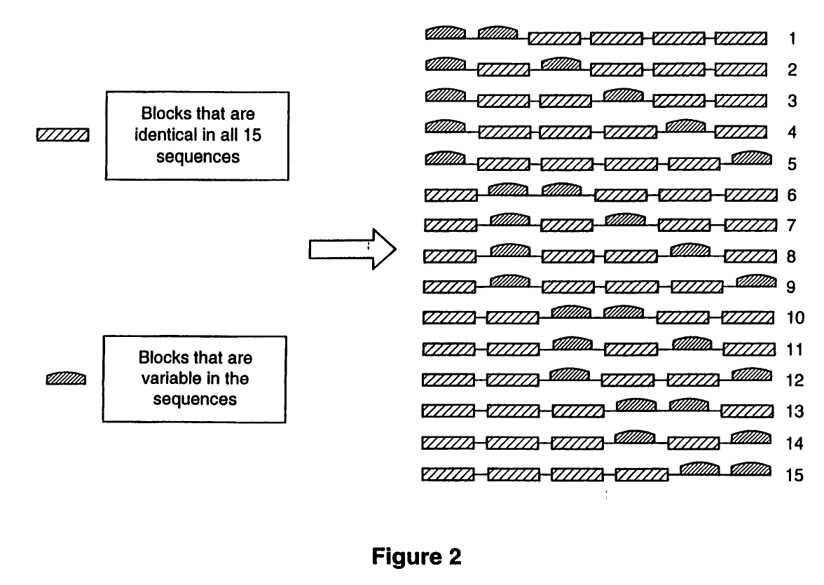

Next, all positions in which each of the block elements can be arranged must be considered. In the example of a 24mer where K=6 and b=4, each topological sequence has six positions, p₁-p₆, in which the block elements K can be arranged. First, all possible topological arrangements of placing the two variable block elements at all six positions are considered. This results in a total of fifteen “topological sequences” (FIG. 2). Of these fifteen, only nine of the topological sequences are considered further. This is because of the dependence of hybridization stability on the total number of base pairs that can form in the resulting polynucleotide duplex. In the example of a 24mer, where K=6 and b=4, if the two polynucleotide strands are perfectly aligned, there are 24 possible base pairs that can contribute to stability of the duplex. By selecting only the nine possible topological sequences that have at least one variable block at the ends, the two interacting strands are forced by favorable interactions in the core to align in such a way that the bases of the variable blocks at the ends do not have any partners for base pairing, and thus do not contribute to duplex stability in a significant manner. This minimizes the overall stability of these topological designs and leaves as possible pairs of strands only those having at most 20 base pairs.

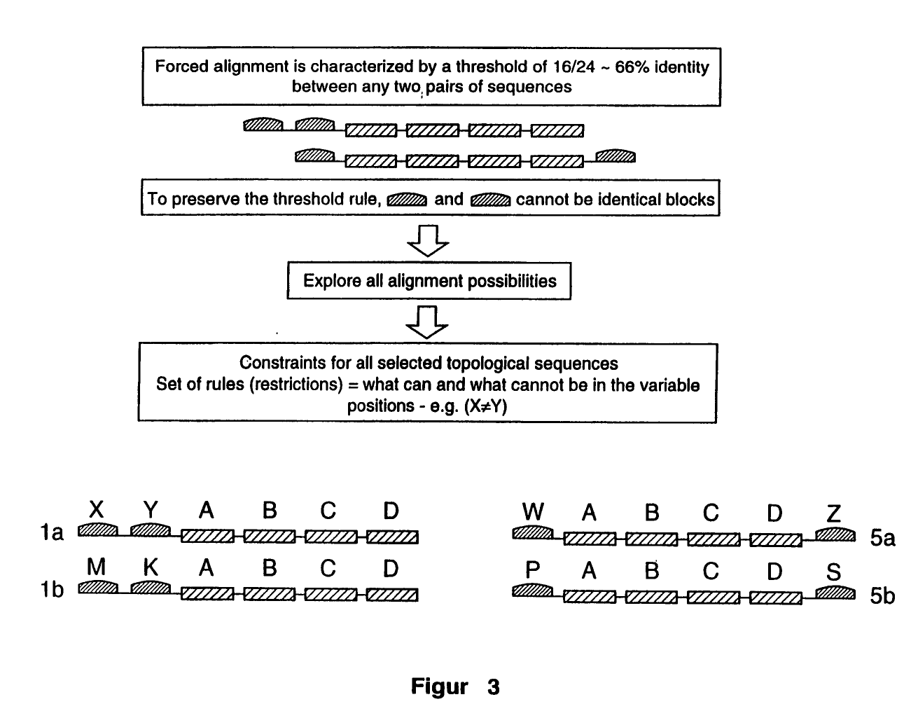

Constraints are now imposed on the topological sequences to force pairwise alignments characterized by the number of core positions allowed to be filled identically between any two pairs of sequences as described above. In this example, for the 24mer, this threshold is set at 66⅔% i.e., any four blocks in the core (16 of 24 nucleotides) can be identical between any two pairs of 24mer polynucleotide sequences (FIG. 3). These constraints are expressed as a set of rules (or restrictions) on the identities of the blocks in the two positions having the variable designation in each sequence. For illustrative purposes, consider the two topological sequences 1 and 5 shown in FIG. 2 with identical blocks A, B, C, and D (FIG. 3). Two sequences for topological sequcence 1 and two sequences for topological sequences 5 are compared. For topological sequence 1, the pair of sequences have the variable blocks X and Y and M and K. For topological sequence 5, the pairs of sequences have for the variable blocks, Wand Z and P and S. By aligning sequences 1a and 1b in topological sequence 1 and sequences 5a and 5b in topological sequence 5, block by block one sequence with respect to the other, first to the left and then to the right, it can be seen that certain restrictions on the identities of the blocks in the variable positions are required in order to ensure four or less blocks are identical between the two sequences i.e., that no more than four identical blocks can occur in any pair. To illustrate, take for example sequences 1a and 1b of topological sequence 1:

| Alignment 1 | Alignment 2 | Alignment 3 |
|---|---|---|
| XYABCD | XYABCD | XYABCD |
| MKABCD | MKABCD | MKABCD |

For Alignment 1, this leads to the restrictions X≠M and Y≠K. For the Alignments 2 and 3 i.e., for sliding to the right or left, there are no further restrictions since no more than four identical block elements occur in either pair. In an analogous manner, all other possible alignments are considered for the chosen set of nine topological sequences. Rules governing the restrictions for all these pairwise alignments are given in Table 1.

TABLE 1
A set of restrictions on the identities of the variable block elements in FIG.
3, which can be obtained by considering all possible pairwise alignment
possibilities between topological sequences 1, 2, 3, 4, 5, 9, 12, 14 and 15 of
FIG. 2. As an example, pairwise alignments between topological sequences
1 and 5 and between 2 and 9 of FIG. 2 are shown.

| | | | | | | | |
|---|---|---|---|---|---|---|---|
| X ≠ M | Y ≠ K | Z ≠ P | W ≠ S | M ≠ X | K ≠ Z | P ≠ A | S ≠ Y |
| X ≠ Y | Y ≠ Z | Z ≠ A | W ≠ Y | M ≠ K | K ≠ P | P ≠ W | S ≠ K |
| | Y ≠ P | Z ≠ W | W ≠ K | | K ≠ A | P ≠ S | S ≠ Z |
| | Y ≠ A | Z ≠ S | W ≠ P | | K ≠ W | P ≠ D | S ≠ W |
| | Y ≠ W | Z ≠ D | W ≠ Z | | K ≠ S | P ≠ Y | S ≠ P |
| | Y ≠ S | Z ≠ Y | | | K ≠ M | P ≠ Z | |
| | Y ≠ X | Z ≠ K | | | K ≠ D | P ≠ K | |
| | Y ≠ D | | | | K ≠ Y | | |

Creating Sequence Templates

To increase the total number of sequences generated, combinations of different topological sequences need to be used. The distribution of block elements in these different topological sequences are to have the following properties:

(a) obey all rules of Table 1, and

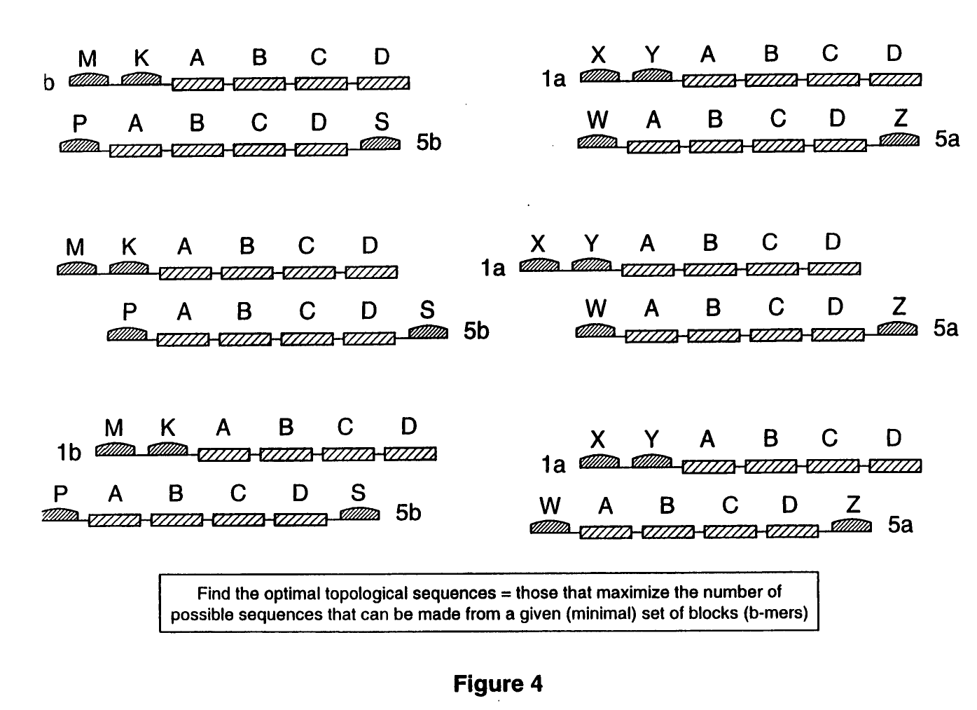

(b) the block sequences generated using any two different topological sequences do not have more than four identical blocks when compared in perfect alignment with no overlay or when shifted by one block element to the right and to the left relative to each other as shown explicitly in FIG. 4 for topological sequences 1 and 5 of FIG. 3. In FIG. 4, topological sequences 1a and 1b are compared with 5a and 5b. This procedure is repeated for all possible pairwise comparisons of all topological sequences in FIG. 2

Results of the comparisons are summarized in Table 2.

TABLE 2
The capacity of each topological sequence and the rules for each specific
sequence template applied for positioning the 1-6 bmers. These are the
sequence templates used.

# of building blocks = 6:

| tetramer | 001111 | 011110 | 111100 | 010111 | 101110 | 111010 | 011101 |
|---|---|---|---|---|---|---|---|
| a = d = i | 150 | 180 | 150 | 150 | 180 | 150 | 180 |
| | xyabcd | xabcdy | abcdxy | xaybcd | axbcdy | abcxdy | xabcyd |
| | x,y = 1 . . . 6 | x,y = 1 . . . 6 | x,y = 1 . . . 6 | x,y = 1 . . . 6 | x,y = 1 . . . 6 | x,y = 1 . . . 6 | x,y = 1 . . . 6 |
| | x ≠ y | x ≠ y | x ≠ y | x ≠ y | x ≠ y | x ≠ y | x ≠ y |
| | y ≠ i | | x ≠ i | y ≠ I | | x ≠ i | |
| a = i | 600 | 630 | 870 | 600 | 390 | 870 | 630 |
| d = j | xyabcd | xabcdy | abcdxy | xyabcd | axbcdy | abcxdy | xabcyd |
| | x,y = 1 . . . 6 | x,y = 1 . . . 6 | x,y = 1 . . . 6 | x,y = 1 . . . 6 | x,y = 1 . . . 6 | x,y = 1 . . . 6 | x,y = 1 . . . 6 |
| | x ≠ y | x ≠ i AND y ≠ j | x ≠ i AND y ≠ j | x ≠ y | x ≠ i AND y ≠ j | x ≠ i AND y ≠ j | x ≠ i AND y ≠ j |
| | y ≠ i, y ≠ j | x ≠ j AND y ≠ I | x ≠ j | y ≠ i, y ≠ j | x ≠ j | x ≠ j | x ≠ j AND y ≠ i |
| | | x ≠ j AND y ≠ j | | | Rule 2 | | x ≠ j AND y ≠ j |
| | | Rule 1 | | | | | Rule 1 |

Rule 1: IF(x ≠ i AND x ≠ j) THEN (y = i OR y = j OR y = x)
Rule 2: IF (x ≠ i AND x ≠ j) THEN (y = i OR y = j)
# of building blocks = 7:

| tetramer | 001111 | 011110 | 111100 | 010111 | 101110 | 111010 | 011101 |
|---|---|---|---|---|---|---|---|
| a = d = i | 216 | 252 | 216 | 216 | 252 | 216 | 252 |
| | xyabcd | xabcdy | abcdxy | xaybcd | axbcdy | abcxdy | xabcyd |
| | x,y = 1 . . . 7 | x,y = 1 . . . 7 | x,y = 1 . . . 7 | x,y = 1 . . . 7 | x,y = 1 . . . 7 | x,y = 1 . . . 7 | x,y = 1 . . . 7 |
| | x ≠ y | x ≠ y | x ≠ y | x ≠ y | x ≠ y | x ≠ y | x ≠ y |
| | y ≠ i | | x ≠ i | y ≠ i | | x ≠ i | |
| a = i | 900 | 1380 | 1230 | 900 | 1080 | 1230 | 1380 |
| d = j | xyabcd | xabcdy | abcdxy | xyabcd | axbcdy | abcxdy | xabcyd |
| | x,y = 1 . . . 7 | x,y = 1 . . . 7 | x,y = 1 . . . 7 | x,y = 1 . . . 7 | x,y = 1 . . . 7 | x,y = 1 . . . 7 | x,y = 1 . . . 7 |
| | x ≠ y | x ≠ i AND y ≠ j | x ≠ i AND y ≠ j | x ≠ y | x ≠ i AND y ≠ j | x ≠ i AND y ≠ j | x ≠ i AND y ≠ j |
| | y ≠ i, y ≠ j | x ≠ j AND y ≠ i | x ≠ j | y ≠ i, y ≠ j | x ≠ j | x ≠ j | x ≠ j AND y ≠ i |
| | | x ≠ j AND y ≠ j | | | Rule 3 | | x ≠ j AND y ≠ j |

# of building blocks = 8:

| tetramer | 001111 | 011110 | 111100 | 010111 | 101110 | 111010 | 011101 |
|---|---|---|---|---|---|---|---|
| a = d = i | 294 | 336 | 294 | 294 | 336 | 294 | 336 |
| | xyabcd | xabcdy | abcdxy | xaybcd | axbcdy | abcxdy | xabcyd |
| | x,y = 1 . . . 8 | x,y = 1 . . . 8 | x,y = 1 . . . 8 | x,y = 1 . . . 8 | x,y = 1 . . . 8 | x,y = 1 . . . 8 | x,y = 1 . . . 8 |
| | x ≠ y | x ≠ y | x ≠ y | x ≠ y | x ≠ y | x ≠ y | x ≠ y |
| | y ≠ i | | x ≠ i | y ≠ i | | x ≠ i | |
| a = i | 1260 | 1830 | 1650 | 1260 | 1470 | 1650 | 1830 |
| d = j | xyabcd | xabcdy | abcdxy | xaybcd | axbcdy | abcxdy | xabcyd |
| | x,y = 1 . . . 8 | x,y = 1 . . . 8 | x,y = 1 . . . 8 | x,y = 1 . . . 8 | x,y = 1 . . . 8 | x,y = 1 . . . 8 | x,y = 1 . . . 8 |
| | x ≠ y | x ≠ i AND y ≠ j | x ≠ i AND y ≠ j | x ≠ y | x ≠ i AND y ≠ j | x ≠ i AND y ≠ j | x ≠ i AND y ≠ j |
| | y ≠ i, y ≠ j | x ≠ j AND y ≠ i | x ≠ j | y ≠ i, y ≠ j | x ≠ j | x ≠ j | x ≠ j AND y ≠ i |
| | | x ≠ j AND y ≠ j | | | Rule 3 | | x ≠ j AND y ≠ j |

Rule 3: IF(x ≠ i AND y ≠ i) THEN x ≠ y

The rules for block selection ensuring that the conditions described above in (a) and (b) are satisfied can be developed as described below. Additional rules can be formulated that reduce the number of steps in the final sequence design. As a practical result of this analysis, “sequence templates” are developed using the topological sequences and rules, which apply to them (see Table 2). A sequence template is a block sequence having assigned to each of its block positions a set of rules for filling that position with a specific fixed designation. (It is possible that the rule is that the block can be filled with any specific fixed designation.) It is a generalization, as far as the sequence templates that are part of the example illustrated herein, that each of blocks designated B and C (see below) can be filled by any of the six specific fixed designations 1 to 6. The combined rules prescribing the selections possible determine the topological capacity of any topological sequence. The “topological capacity” is defined here as the maximal number of sequences (ignoring positions B and C) that can be generated for a given topological sequence using the rules that apply to that topological sequence. The capacity of any topological sequence can be evaluated and the most potent topological sequences can thus be selected for combination in to a sequence template set to ensure the maximal efficiency of the sequence design method.

By way of illustration, the process of generating the additional rules shown in Table 2, for template capacity determination using the rules from Table 1, for the template design X A B C D Y, with positions B C permitted to have any value is shown generally below:

STEP 1: Generate all I sequences from the set of K (6) available bmers using the template design X A B C D Y using the following algorithm:

I = 1,
For i 1 = 1 to K

For i 2 = 1 to K

For i 3 = 1 to K

:
:
:
For i 6 = 1 to K

Sequence [I] = b[i 1] + . . . + b[i 6]
I = I + 1
END
:
:
END

I = I − 1

STEP 2: Once all the sequences have been generated by the algorithm in Step 1, those sequences that violate the rules of Table 1 are determined by a simple selection algorithm and are not considered further. Thus,

For i=1 to I,

If all the rules from Table 1 applied to sequence [i] are not violated, then write sequence [i] into OUTPUT. ELSE skip sequence [i]

END

STEP 3: All the sequences written into OUTPUT are read, and the position of the B C string in these sequences identified. This defines positions for the i and j block elements. The bmers, which can be placed in positions i and j are next identified. Thus,

For i1=1 to 6 (number of bmers)

For j1=1 to I

If bmer in position i is identical to bmer in position j—THEN WRITE the sequence j1 into the OUTPUT 011110ii[j1]—ELSE SKIP sequence [j1]

If bmer in position i is NOT identical to bmer in position j—THEN WRITE the sequence [j1] into OUTPUT 011110ij [i1]—ELSE SKIP SEQUENCE [j1]

END

END

STEP 4: The OUTPUT from the previous step is analyzed and all the sequences in the outputs for all template types, for cases ii and ij are counted. This is how the template capacity is determined. The results of the complete analysis of all possibilities for the various sequence template types can be summarized in the form of the general rules given in Table 2 for the sequence templates. In other words, the rules in Table 2 are a summary of the complete evaluation of all possibilities of placing all bmers in the respective positions of the respective sequence templates as follows from step 4 above. The rules shown in Table 2, for each sequence template cannot be simply formulated without software assisted evaluation of all possibilities according to the rules of Table 1, then sorting out the remaining sequences according to their template topology and template “sub-type” (i.e., ii and ij). This method does not necessarily guarantee that all sequences generated from the templates shown in Table 2 will also obey all the rules in Table 1. That is the capacities shown in Table 2 are upper limits. The stated rules and capacities in Table 2 are general for all possibilities of i and j. However, specific values of i and j can lead to specific sequences that violate the rules in Table 1.

Examples generation of a family of block sequences using the rules shown in Table 2 are given below. For the sequence template, X Y A B C D, where A=D=i, the sequence template can be written as,

X Y i B C i (1)

At this point, the blocks in the core center positions, B and C, remain unspecified. Thus, B and C can be represented as blanks, and sequence template (1) can now be re-written as,

X Y i - - i (2).

The template capacity is next determined by considering all possible combinations of X and Y and i bmers without any regard to the rules in Table 1. All possible sequence combinations are first listed, after which those sequences violating the rules in Table 1 are deleted. Thus, where X=1, Y=1, 2, 3, 4, 5, or 6, and where i=1, the possible sequence combinations, which can be generated without any regard to the rules established in Table 1 are:

| X | Y | i | — | — | i | |
|---|---|---|---|---|---|---|
| 1 | 1 | 1 | — | — | 1 | (3) |
| 1 | 2 | 1 | — | — | 1 | (4) |
| 1 | 3 | 1 | — | — | 1 | (5) |
| 1 | 4 | 1 | — | — | 1 | (6) |
| 1 | 5 | 1 | — | — | 1 | (7) |
| 1 | 6 | 1 | — | — | 1 | (8) |

Similarly, where X=2, Y=1, 2, 3, 4, 5, or 6, and where i=1, the possible sequence combinations which can be generated without any regard to the rules in Table 1 are:

| X | Y | i | — | — | I | |
|---|---|---|---|---|---|---|
| 2 | 1 | 1 | — | — | 1 | (9) |
| 2 | 2 | 1 | — | — | 1 | (10) |
| 2 | 3 | 1 | — | — | 1 | (11) |
| 2 | 4 | 1 | — | — | 1 | (12) |
| 2 | 5 | 1 | — | — | 1 | (13) |
| 2 | 6 | 1 | — | — | 1 | (14) |

The above can be used generate the following possible sequence combinations for each X=3, 4, 5, and 6, again without any regard to the rules in Table 1:

| X | Y | i | — | — | I | |
|---|---|---|---|---|---|---|
| 3 | 1 | 1 | — | — | 1 | (15) |
| 3 | 2 | 1 | — | — | 1 | (16) |
| 3 | 3 | 1 | — | — | 1 | (17) |
| 3 | 4 | 1 | — | — | 1 | (18) |
| 3 | 5 | 1 | — | — | 1 | (19) |
| 3 | 6 | 1 | — | — | 1 | (20) |
| X | Y | i | — | — | i | |
| 4 | 1 | 1 | — | — | 1 | (21) |
| 4 | 2 | 1 | — | — | 1 | (22) |
| 4 | 3 | 1 | — | — | 1 | (23) |
| 4 | 4 | 1 | — | — | 1 | (24) |
| 4 | 5 | 1 | — | — | 1 | (25) |
| 4 | 6 | 1 | — | — | 1 | (26) |
| X | Y | i | — | — | i | |
| 5 | 1 | 1 | — | — | 1 | (27) |
| 5 | 2 | 1 | — | — | 1 | (28) |
| 5 | 3 | 1 | — | — | 1 | (29) |
| 5 | 4 | 1 | — | — | 1 | (30) |
| 5 | 5 | 1 | — | — | 1 | (31) |
| 5 | 6 | 1 | — | — | 1 | (32) |
| X | Y | i | — | — | i | |
| 6 | 1 | 1 | — | — | 1 | (33) |
| 6 | 2 | 1 | — | — | 1 | (34) |
| 6 | 3 | 1 | — | — | 1 | (35) |
| 6 | 4 | 1 | — | — | 1 | (36) |
| 6 | 5 | 1 | — | — | 1 | (37) |
| 6 | 6 | 1 | — | — | 1 | (38) |

Thus, 36 possible sequence combinations can be listed without any regard to the rules shown in Table 1. However, some of the above possible sequence combinations violate some of the rules in Table 1. The rules for the above template are that X≠Y and Y≠A.

The possible sequence combinations that violate these two rules that must be deleted are sequences (3), (9), (10), (15), (17), (21), (24), (27), (31), (33) and (38). This leaves 900 be generated from this template

For analysis of the sequence template capacity, the core positions in the topological sequences are divided in the following ways:

(c) the boundary positions (A and D in Table 1) are specified by the rules for determining allowed blocks in these two boundary positions in various sequences according to the rules shown in bold italics in Table 1.

(d) the inner positions (B and C in Table 1) in which the selection of the blocks in various sequences are unconstrained.

Thus there are two subsets of sequence templates that generate well-behaved subsets of polynucleotide sequences (Table 2) these are sequence templates, which have:

(e) type ii-cores where the bmers in the boundary positions are identical i.e., positions A and D are filled with the same bmers and

(f) type ij-cores where the blocks in the boundary positions are different i.e., positions A and D are filled with different bmers.

Determination of the constraints on the allowed bmers at the various positions of the sequence templates consists of the following steps:

(g) generation of all sequences for all topological sequences using the restriction rules summarized in Table 1. bmers in the core center positions of the topological design i.e., bmers B and C, are unspecified,

(h) decomposition of the set of such sequences into subsets characterized by the topological sequences and the ii- and ij-types of cores,

(i) within each subset generated in step (h), the core and variable parts are separated, listed, and the bmer types allowed in each topological sequence are evaluated, and

(j) results of the previous step are compiled and summarized in Table 2 as the rules defining the constraints on the bmer selection in the prescribed positions of the sequence templates.

Determination of the template capacity and selection of the final sequence templates for sequence generation utilizes the above-defined constraints and is performed considering separately the variable block sequences with ii- and ij-type cores, thus:

(k) for the ii-core type of any particular topological sequence, fill the boundary core positions systematically with all K available block elements;

(l) for every partial sequence generated in the previous step, fill the positions in the variable part with b-mers using the rules shown in Table 2. The number of sequences thus generated is the ii-sequence template capacity, which is shown in the upper left hand corner of each template in Table 2. Thus, the template capacity for the template XYABCD, where A=D=i for each i=1-6, is 150 (ignoring positions B and C).

(m) select sequence templates that yield the largest number of sequences with their prescribed b-mers in appropriate positions in the sequence. This choice restricts the selection of templates with the ii-core type;

(n) for the ij-core types repeat the above steps. Similarly, the template capacity for the template ABCDXY, where A=i and D=j, i≠j for each i=1-6 and for each j=1-6, is 870 (ignoring positions B and C).

(o) identify all sequence templates that generate the largest number of sequences;

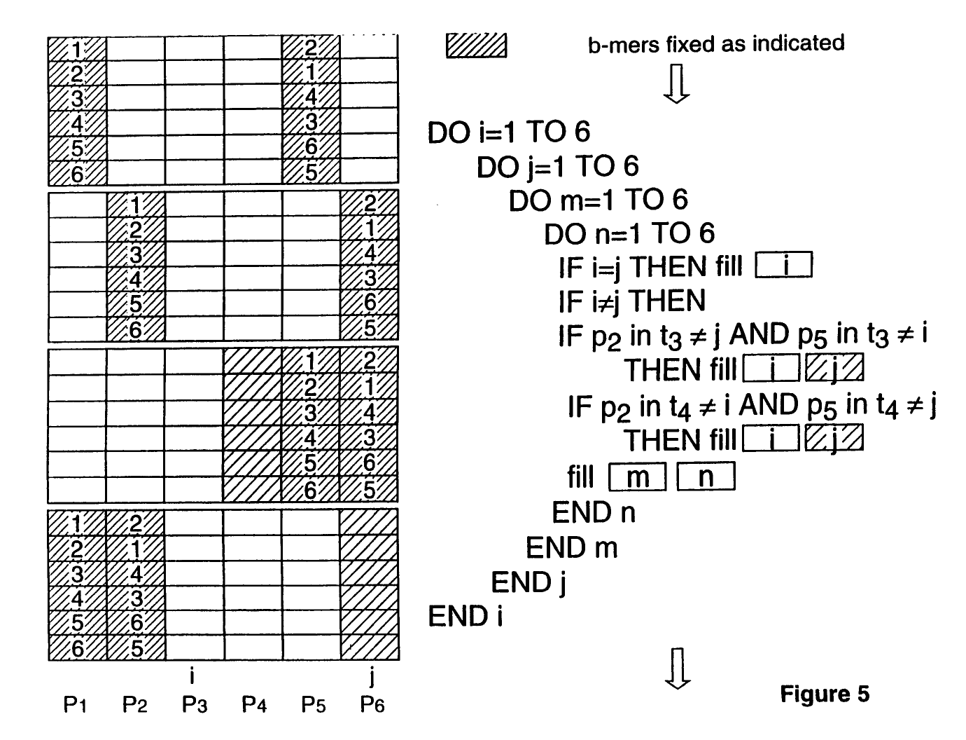

(p) four selected sequence templates are used in the sequence generating algorithm shown in FIG. 5.

Application of the sequence templates and the sequence generating algorithm reduces all total possible sequences, T to a reduced set of topologically acceptable sequences, TA. The number of acceptable sequences TA is ˜15,000 sequences.

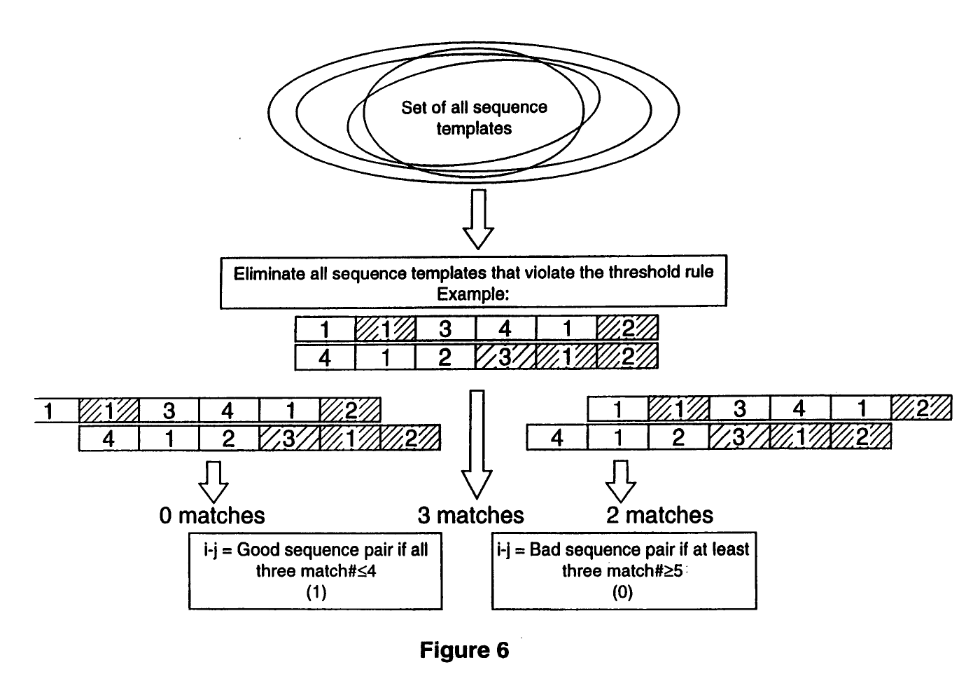

Due to the lack of constraints on the core blocks B and C, the above selected sequences could still have more than four common blocks, therefore these approximately 15,000 sequences are compared pairwise: st, s, t=1, 2, 3, 4, . . . , 15,000 when they are perfectly aligned, shifted one block to the left and one block to the right resulting in ½*3*15,000*(15,000−1)=337,477,500 pairwise comparisons (FIG. 6). If for any sequence pair, st, in any of the three alignments there are more than four blocks in common then that sequence pair is assigned a ‘0’. If there are four or less common blocks in any of the three alignments of the sequences s and t, then that pair is assigned a ‘1’. To better understand how the lack of constraints on the core blocks B and C can still have more than the threshold common blocks, it will be beneficial to go through an example of one template sequence shown in Table 2. For the sequence template, A B CD X Y, where A=s, D=t, the sequence template can be written as,

i B C j X Y (39).

Since, the blocks in the core center positions, B and C, remain unspecified, B and C can be represented as blanks, and sequence template (39) can be written as,

i - - j X Y (40).

In the case where i=block element 3 and j=block element 2, and where X=block element 1 and Y=block elements 1, 2, 3, 4, 5 or 6, the possible sequence combinations, which can be generated are:

| i | — | — | j | X | Y | |
|---|---|---|---|---|---|---|
| 3 | — | — | 2 | 1 | 1 | (41) |
| 3 | — | — | 2 | 1 | 3 | (42) |
| 3 | — | — | 2 | 1 | 4 | (43) |
| 3 | — | — | 2 | 1 | 5 | (44) |
| 3 | — | — | 2 | 1 | 6 | (45) |

At this point, sequence (41)-(45) can all be considered good sequences in that the sequences do not violate the rules of Table 1 and the rules in Table 2 for this particular template. However, once B and C are assigned as one of the block elements 1-6, some of the sequences (41)-(45) could still have more than the threshold number of common blocks. One sequence in particular is sequence (42). Thus, if B is assigned block element 1 and C is assigned any one of block elements 1-6 in sequence (42), the following sequences are generated:

| | | | | | | |
|---|---|---|---|---|---|---|
| 3 | B | C | 2 | 1 | 3 | (42) |
| 3 | 1 | 1 | 2 | 1 | 3 | (46) |
| 3 | 1 | 2 | 2 | 1 | 3 | (47) |
| 3 | 1 | 3 | 2 | 1 | 3 | (48) |
| 3 | 1 | 4 | 2 | 1 | 3 | (49) |
| 3 | 1 | 5 | 2 | 1 | 3 | (50) |
| 3 | 1 | 6 | 2 | 1 | 3 | (51) |

It can be seen that by assigning block element 1 to position B, all sequences (46)-(51) will exceed the set threshold of having more than four common blocks i.e., the set threshold of 66%, regardless of which block elements are placed at position C.

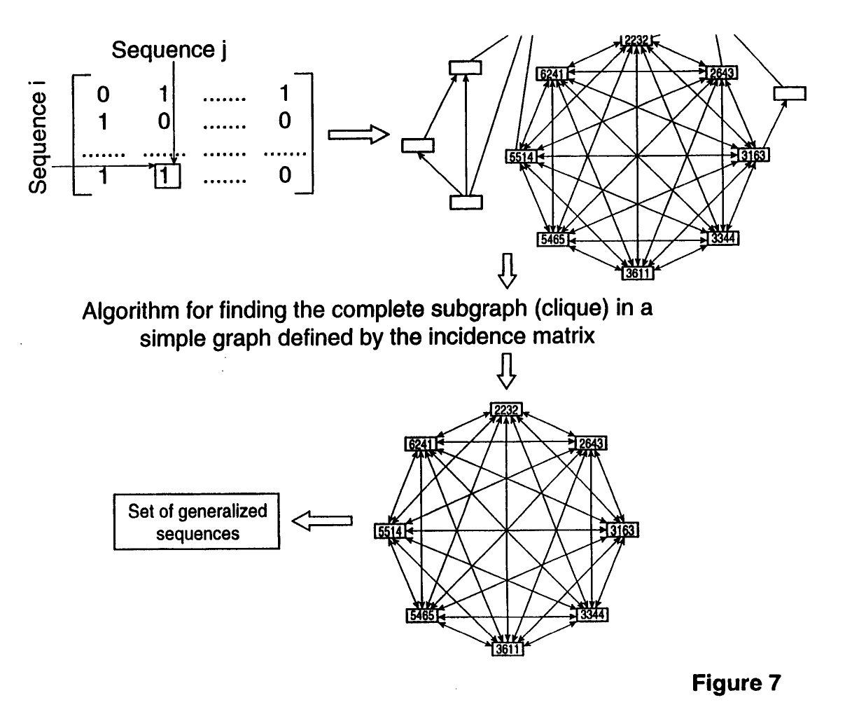

Once the pairwise comparisons are made to eliminate the sequences that exceed the 66% threshold, an incidence matrix is constructed of rows of sequence s=1 to ˜15,000 and columns of sequence t=1 to ˜15,000, with elements of 0 or 1 as described above for all the remaining sequences (FIG. 7).

The incidence matrix is next represented as a simple graph where the vertices are the ˜15,000 sequences and edges connect pairs of sequences with elements equal to 1. An algorithm is applied to find the desired class property, that is, when compared pairwise, all sequences within a set have four or less blocks in common. These sequences are found from the complete subgraphs or “cliques” of the simple graph generated from the incidence matrix. For the simple graph generated from a given incidence matrix there may be more than one clique (FIG. 7). A certain number of cliques are selected.

Next, the sequences in each selected clique are tabulated and counted. Sequences comprising each clique have the desired class property. Although sequences from each clique have the desired property, sequences from different cliques do not necessarily share the desired property. Preliminary results indicate that it should be possible to obtain sets of at least several hundred sequences.

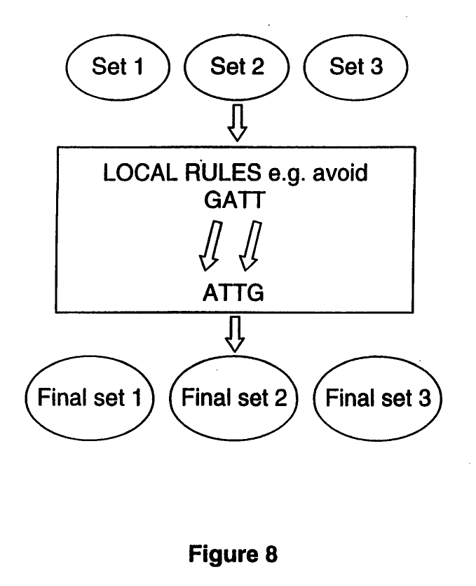

For the set of sequences from each clique, the “local rules” are applied as shown in FIG. 8. This amounts to pairwise comparison of every sequence in the set with every other sequence in the set and moving each one sequence with respect to the other one base at a time and counting the number of common bases. As earlier, these comparisons provide an incidence matrix where an element of 0 is assigned to each pair exceeding the threshold of common nucleotides (16 in the example), and an element of 1 is assigned otherwise. The incidence matrix represents a graph, where vertices correspond to sequences, in which cliques are selected.

The resulting sequences are tabulated. If the final number of sequences obtained is sufficient then the sequence selection and design is completed. However, if an insufficient number of sequences is obtained from K=6 tetramers (for example), then the number can be increased, for example, by use of an additional bmer block with a different sequence than the other blocks is and the process repeated until the desired number of sequences are generated.

If the final set of sequences has to satisfy further similarity conditions, for example involving insertions and deletions, it is possible to add further filtering steps analogous to the previous ones. More precisely, another condition can be rejection of a sequence which, when paired with another, has more than 19 bases in common with the other when alignments with insertions/deletions are performed. In this case, an incidence matrix is created by performing all possible pairwise alignments with insertions/deletions, putting a 0 in an entry of the matrix if the total number of matches for the corresponding pair exceeds 19, and a 1 otherwise. Only the sequences corresponding to a clique of the associated graphs would be kept.

Looking at Table 2, the number of block different block sequences that can be produced based on the template 001111, and for i=j can be determined using its sequence capacity of 150. Here the number of different block sequences is 150×6=900 if six different specific blocks are used to construct a family of block sequences. As described above, not all of these sequences will have the desired property of having a simple homology of no more that 66⅔% with any other sequence. Cliques of block sequences in which all members of the clique have the property are obtainable, as described above.

Once a clique of block sequences is determined, then the tetramers (or dimers, trimers, pentamers, etc., as may be desired) can be used to create a family of nucleotide sequences. Again, there may be sequences created that do not have the desired property of having no more than 66⅔% homology with every other member of the family. As with the block sequences, cliques of polynucleotide sequences wherein each sequence within such a clique has the desired property can be created by shifting pairs of aligned fragments with respect to each other, as described above. A person skilled in the art would of course understand that the generation of one or more cliques of block sequences, can be omitted. That is, it would be possible to create a group of nucleotide sequences directly using a template and rules of Table 2 and then to generate a clique (subset) of sequences, all of which members of the clique have the desired property.

In any event, another aspect of this invention is a method obtaining a group of polynucleotide sequences wherein each member fragment of the group shares no more than a given degree of simple homology with each other member of the group.

According to the disclosed embodiment, the given degree of simple homology is 66⅔% and final nucleotide sequences are based on a family of block sequences six blocks in length. There are many possibilities. For example, one might base the method on a block sequence having ten blocks and selecting a simple homology of 10, 20, 30, 40, 50, 60, 70, 80 or 90%. One might base the method on a block sequence having 20 blocks and selecting a simple homology of 5, 10, 15, 20, 25, 30, 35, 40, 45, 50, 55, 60, 65, 70, 75, 80, 85, 90 or 95%. Shared homologies of no more than about 60 or 70 percent are likely to be more preferred, particularly if the desired property to be manifest in a primary final group (clique) of nucleotide fragments is to be reduced cross-hybridization of the non-matching complementary sequences (each member of the family of complementary sequences, of course, being complementary to one of the primary group).

The length of each member sequence of a clique of polynucleotide sequences is a function of both the length of the building blocks (1 (single base or monomer), 2 (dimer), 3 (trimer), 4 (tetramer), 5 (pentamer), etc. to any number of nucleotides per building block that it is possible from which to build longer nucleic acid molecules) and the number of building blocks (2, 3, 4, 5, 6, 7, 8, 9, etc.) used to create the family, and ultimately, the clique(s) of polynucleotide sequences, which correspond to the sequences of nucleic acid molecules eventually to be synthesized.

According to this invention, a given clique or grouping can have at least 100, or at least 200, or at least 300, or at least 400, or at least 500, or at least 600, or at least 700, or at least 800, or at least 900, or at least 1000, or at least 1100, or at least 1200, or at least 1300, or at least 1400, or at least 1500, or at least 1600, or at least 1700, or at least 1800, or at least 1900, or at least 2000, or at least 2100, or at least 2200, or at least 2300, or at least 2400, or at least 2500, or at least 2600, or at least 2700, or at least 2800, or at least 2900, or at least 3000, or at least 3100, or at least 3200, or at least 3300, or at least 3400, or at least 3500, or at least 3600, or at least 3700, or at least 3800, or at least 3900, or at least 4000, or at least 4100, or at least 4200, or at least 4300, or at least 4400, or at least 4500, or at least 4600, or at least 4700, or at least 4800, or at least 4900, or at least 5000, or at least 5100, or at least 5200 or more member polynucleotide fragments.

A clique or group of polynucleotide fragments, in which each member fragment of the group shares no more than a limited degree of simple homology with each other member of the group, as according to the current invention, finds utility when used found to minimally cross-hybridize with each other, for example as when bound to “zip chip” as described above. Preferably, each nucleic acid molecule fragment of the family has at least about ten nucleotides, more preferably at least 15, or about 20 or more, or 24 or more. Preferably, there are at least about 1000 fragments, more preferably about 2000 or more, 3000 or more or 4000 or more.

Preferably, the fragments of a given family are of the same length as each other.

Polynucleotides of a given family preferably have similar compositions to each other, or as it is known in the art, to have a similar “G-C content”. This should lead to a clique of polynucleotides in which the melting temperatures (Tₘ) of the members and their complements are similar to one another. In the case of sequences built using building blocks of more than one nucleotide base (i.e., dimer, trimer, tetramer, etc., which ever polymer length is used) preferably, the polymers are chosen so as to maximize the number of members in a clique.

It will be understood that once a clique of polynucleotide fragments is determined for use in hybridization methods, it will often be necessary to optimize the conditions under which such hybridization is to be conducted. That is, the conditions can be adjusted so as to increase the chances that a sequence and its complement will hybridize with each other and that a sequence will not hybridize with a sequence that is not precisely complementary to it. Such optimization, which would involve trying different salt concentrations, different temperatures, etc., is within the ability of a person skilled in the art.

The polynucleotide sequences generated by the method described above can be used for generating probes on an array or beads. There are several ways of making an array and fall generally into three categories: In situ or on-chip syntheses of oligonucleotides; arraying of prefabricated oligonucleotides; and spotting of polynucleotide fragments.

Two approaches have been used for in situ polynucleotide synthesis. Affymetrix fabricates polynucleotide arrays on the chip using photolithography. In this method, a mercury lamp is shone through a photolithographic mask onto the chip surface, which removes a photoactive group, resulting in a 5′ hydroxy group capable of reacting with another nucleoside. The mask therefore predetermines which nucleotides are activated. Successive rounds of deprotection and chemistry result in oligonucleotides up to 30 bases in length.

Another approach, the piezoelectric printing method, uses technology analogous to that currently employed in “ink-jet” printers. Here, the printer “head” travels across the array, and at each spot, electric current expands an adapter, encircling a tube containing the reagents for one of the four bases, forcing a microliter drop of the reagent onto the coated surface, where it is anchored using standard chemistry. Following washing and deprotection, the next cycle of oligonucleotide synthesis is carried out. Oligonucleotide lengths of 40-50 bases are possible.

Another way of making arrays is to “spot” cDNAs directly onto the chip surface. Glass slides are overlayed with a positively charged coating, such as amino silane or polylysine, and polynucleotide fragments suspended in the denaturing solution are then printed directly onto the surface.

All references described herein are incorporated into the specification by reference, including the U.S. Priority Application No. 60/181,563, filed Feb. 10, 2000.

A person skilled in the art will appreciate that the preferred embodiments described herein can be varied while remaining within the scope of the invention, the ambit of which is defined by the following claims.

1. A method of processing a family of topological block sequences useful in creating a family of nucleic acid molecules, the method comprising:
(a) providing first and second topological block sequences, each sequence having a predetermined number of core blocks and a predetermined number of variable blocks;
(b) aligning the first and second sequences with each other such that at least one block of the first sequence is paired with at least one block of the second sequence in an aligned arrangement;
(c) assigning conditions to the variable blocks of the first and second sequences as necessary to provide that, in the arrangement of (b), the sum of (i) the number of pairs of aligned core blocks, and (ii) the number of pairs of aligned variable blocks, in which both variable blocks are permitted to have the same designation, does not exceed a predetermined threshold; and
(d) storing the conditions assigned in (c) for each variable block of the first and second sequences in a computer readable medium in association with the respective first and second sequences.

2. The method of claim 1, wherein a said step (a) includes providing said first and second topological sequences which have the same topology as each other, and the first and second sequences are aligned with each other such that each core block of one sequence is paired with a core block of the other sequence.

3. The method of claim 1, wherein a said step (a) includes providing said first and second topological sequences having topologies different one from the other, and the first and second topological sequences are aligned with each other such that the number of pairs of aligned core blocks is maximized.

4. The method of claim 3, wherein a said step (a) includes providing said first and second topological sequences which have the same topology as each other, and the first and second sequences are aligned with each other such that each core block of one sequence is paired with a core block of the other sequence.

5. The method of claim 2, further comprising the steps of:
(e) providing a database of specific block sequences;
(f) determining which of the plurality of specific block sequences meet the conditions assigned in step (c); and
(g) storing the specific block sequences determined in step (f) to meet the conditions assigned in step (c) into a database.

6. The method of claim 3, further comprising the steps of:
(e) providing a database of specific block sequences;
(f) determining which of the plurality of specific block sequences meet the conditions assigned in step (c); and
(g) storing the specific block sequences determined in step (f) to meet the conditions assigned in step (c) into a database.

7. The method of claim 4, further comprising the steps of:
(e) providing a database of specific block sequences;
(f) determining which of the plurality of specific block sequences meet the conditions assigned in step (c); and
(g) storing the specific block sequences determined in step (f) to meet the conditions assigned in step (c) into a database.

8. The method of claim 1, wherein each topological block sequence comprises at least five blocks and at least three of the blocks are core blocks.

9. The method of claim 2, wherein each topological block sequence comprises at least five blocks and at least three of the blocks are core blocks.

10. The method of any of claims 3 to 7, wherein each topological block sequence comprises at least five blocks and at least three of the blocks are core blocks.

11. The method of claim 1, further comprising the step of:
(h) repeating steps (b) through (d) for a different said aligned arrangement of pairs of topological block sequences, having topologies different one from the other, of step (b).

12. The method of any of claims 3 to 8, or claim 10, further comprising the step of:
(h) repeating steps (b) through (d) for a different said aligned arrangement of said first and second topological block sequences having topologies different one from the other, of step (b).

13. The method of claim 1, further comprising the step of:
(i) repeating steps (b) through (d) for a different pair of first and second topological block sequences.

14. The method of claim 2, further comprising the step of:
(i) repeating steps (b) through (d) for a different pair of said first and second topological block sequences which have the same topology as each other.

15. The method of claim 3, further comprising the step of:
(i) repeating steps (b) through (d) for a different pair of first and second topological block sequences.

16. The method of any of claims 4 to 10, further comprising the step of:
(i) repeating steps (b) through (d) for a different pair of said first and second topological block sequences which have the same topology as each other.

17. The method of claim 11, further comprising the step of:
(i) repeating steps (b) through (d) and (h) for a different pair of said first and second topological block sequences having topologies different one from the other, of step (b).

18. The method of claim 12, further comprising the step of:
(i) repeating steps (b) through (d) and (h) for a different pair of said first and second topological block sequences having topologies different one from the other, of step (b).

19. The method of claim 12, wherein:
first and second of said core blocks of each topological sequence of the family are each located adjacent a said variable block, and
further comprising the step of:
(j) prior to step (c), assigning to the first and second core blocks, the condition that the first and second core blocks each have the same designation.

20. The method of claim 13, wherein:
first and second of said core blocks of each topological sequence of the family are each located adjacent a said variable block; and
further comprising the step of:
(j) prior to step (c), assigning to the first and second core blocks, the condition that the first and second core blocks each have the same designation.

21. The method of claim 14, wherein:
first and second of said core blocks of each topological sequence of the family are each located adjacent a said variable block; and
further comprising the step of:
(j) prior to step (c), assigning to the first and second core blocks, the condition that the first and second core blocks each have the same designation.

22. The method of any of claims 15 to 18, wherein:
first and second of said core blocks of each topological sequence of the family are each located adjacent a said variable block; and
further comprising the step of:
(j) prior to step (c), assigning to the first and second core blocks, the condition that the first and second core blocks each have the same designation.

23. The method of claim 12, wherein:
first and second of said core blocks of each topological sequence of the family are each located adjacent a said variable block; and
further comprising the step of:
(j) prior to step (c), assigning to the first and second core blocks, the condition that the first core block has a different designation from the second core block.

24. The method of claim 13, wherein:
first and second of said core blocks of each topological sequence of the family are each located adjacent a said variable block; and
further comprising the step of:
(j) prior to step (c), assigning to the first and second core blocks, the condition that the first core block has a different designation from the second core block.

25. The method of claim 14, wherein:
first and second of said core blocks of each topological sequence of the family are each located adjacent a said variable block; and
further comprising the step of:
(j) prior to step (c), assigning to the first and second core blocks, the condition that the first core block has a different designation from the second core block.

26. The method of any of claims 15 to 18, wherein:
first and second of said core blocks of each topological sequence of the family are each located adjacent a said variable block; and
further comprising the step of:
(j) prior to step (c), assigning to the first and second core blocks, the condition that the first core block has a different designation from the second core block.

27. The method of any preceding claim, wherein at least one variable block of each topological sequence of the family is located in a terminal position of a said topological sequence.

28. The method of any of claims 5, 6 or 7, wherein the number of blocks in each topological sequence equals the number of blocks in every other topological sequence and the number of blocks in every specific block sequence, and comprising the further steps of:
(k) assigning an x-mer to each specific designation of a block of each specific block sequence of step (g) with the condition that each block with a first specific designation is assigned an x-mer different from every other block not having the first specific designation;
(l) storing the sequences obtained in step (k) into a database;
(m) selecting a first sequence from the database of step (l)
(n) selecting a second sequence from the database of step (l);
(o) aligning the first and second sequences so as to maximize the number of nucleotides having the same designation;
(p) determining the number of matching pairs of nucleotides;
(q) arranging the first and second sequences of step (p) in a matrix stored in a database, wherein: (r)(i) if the number of matching paired nucleotides is less than or equal to a pre-selected number, then the first and second sequences are associated with each other in the matrix; and (r)(ii) if the number of matching paired nucleotides having the same designation is greater than the pre-selected number, then the first and second sequences are non-associated with each other in the matrix; and (s) repeating steps (m) to (q) for a different pair of first and second sequences so as to form one or more groups of sequences in which each group consists of a set of nucleotide sequences wherein each sequence is associated with every other sequence.

29. A method of processing a family of topological block sequences useful in creating a family of nucleic acid molecules, the method comprising:
(a) providing a first pair of first and second topological block sequences, each sequence having c core blocks and v variable blocks, c and v being natural numbers, the first and second topological sequences each having a first topology,
(b) aligning the first and second sequences with each other such that each core block of one sequence is paired with a core block of the other sequence and each variable block of one sequence is paired with a variable block of the other sequence;
(c) assigning conditions to the variable blocks of the first and second sequences that are necessary to provide that the sum of (i) the number of pairs of aligned core blocks, and (ii) the number of pairs of aligned variable blocks, in which both variable blocks are permitted to have the same designation, does not exceed a predetermined threshold; and
(d) storing the conditions determined for each variable block of the first and second sequences in a computer readable medium in association with the respective first and second sequences.

30. The method of claim 29, further comprising, providing a second pair of first and second topological block sequences, each sequence having c core blocks and v variable blocks, the topological sequences of the second pair each having a second topology, and repeating steps (b) through (d) for the second pair of first and second topological block sequences.

31. The method of claim 30, further comprising:
(e) providing a database of specific block sequences;
(f) determining which of the plurality of specific block sequences meet the conditions assigned in step (c); and
(g) storing the specific block sequences determined in step (f) to meet the conditions assigned in step (c) into a database.

32. The method of claim 30, further comprising:
(1) providing a third pair of first and second topological block sequences, each sequence having c core blocks and v variable blocks, wherein the topological sequences have different topologies one from the other and wherein the topology of one said sequence is the same as the topology of one of the first and second pairs of topological sequences and wherein the topology of the other said sequence is the same as the topology of the other for the first and second pairs of topological sequences;
(2) aligning the first and second topological block sequences provided in step (1) with each other such that the number core blocks in paired alignment with each other is maximized; and
(3) assigning conditions to the variable blocks of the first and second sequences of step (2) that are necessary to provide that the sum of (1) the number of pairs of aligned core blocks, and (2) the number of pairs of aligned variable blocks, in which both variable blocks are permitted to have the same designation, does not exceed the predetermined threshold; and
(4) storing the conditions determined for each variable block of the first and second sequences in a computer readable medium in association with first and second sequence templates, respectively, corresponding to the respective first and second topological sequences.

33. The method of any of claims 29 to 32 wherein the sum of c and v is at least five.

34. The method of claim 33, where the sum of c and v is six.

35. The method of claim 33 or 34, wherein each of v and c is at least two.

36. The method of claim 35, wherein v is two.

37. The method of any of claims 33 to 36, wherein at least one variable block of each topological sequence is located in a terminal position of the topological sequence.

38. The method of any of claims 35 to 37, comprising the further step of, prior to step (3), assigning to the first and second core blocks, the condition that the first and second core blocks each have the same designation.

39. The method of claim 38, further comprising, for each specific block sequence stored in step (g) of claim 31, determining whether the block sequence meets the conditions stored in step (4) of claim 32 in association with a first said sequence template; and storing said sequences into a database.

40. The method of claim 39, further comprising the step of determining the maximum number of specific block sequences that meet the conditions stored in step (4) of claim 32 in association with the first said sequence template.

41. The method of claim 38, further comprising, for each specific block sequence stored in step (g) of claim 31, determining whether the block sequence meets the conditions stored in step (4) of claim 32 in association with a second said sequence template; and storing said sequences into a database.

42. The method of claim 41, further comprising the step of determining the maximum number of specific block sequences that meet the conditions stored in step (4) of claim 32 in association with the second said sequence template.

43. The method of any of claims 35 to 37, comprising the further step of, prior to step (3), assigning to the first and second core blocks, the condition that the first and second core blocks have different designations, one from the other.

44. The method of claim 43, further comprising, for each specific block sequence stored in step (g) of claim 31, determining whether the block sequence meets the conditions stored in step (4) of claim 32 in association with a first said sequence template; and storing said sequences into a database.

45. The method of claim 44, further comprising the step of determining the maximum number of specific block sequences that meet the conditions stored in step (4) of claim 32 in association with the first said sequence template.

46. The method of claim 43, further comprising, for each specific block sequence stored in step (g) of claim 31, determining whether the block sequence meets the conditions stored in step (4) of claim 32 in association with a second said sequence template; and storing said sequences into a database.

47. The method of claim 41, further comprising the step of determining the maximum number of specific block sequences that meet the conditions stored in step (4) of claim 32 in association with the second said sequence template.

48. The method of claim 37, further comprising the steps of (h) selecting a first sequence from the database of step (g) claim 31; (i) selecting a second sequence from the database of step (g) of claim 31; (j) aligning the first and second sequences so as to maximize the number of paired blocks having the same designation; (k) determining the number of matching pairs; (l) arranging the first and second sequences of step (j) in a matrix, wherein: (l)(i) if the number of paired blocks having the same designation is less than or equal to the threshold of step (c) of claim 29, then the first and second sequences are associated with each other in the matrix; and (l)(ii) if the number of paired blocks having the same designation is greater than the threshold of step (c) of claim 29, then the first and second sequences are non-associated with each other in the matrix; and (m) repeating steps (h) to (l) for a different pair of first and second sequences so as to form one or more cliques or groups of sequences, each clique (group) comprising a set of sequences wherein each sequence is associated with every other sequence.

49. The method of claim 48, further comprising, for a said clique: (A) assigning a nucleotide or an x-mer to each specific designation to obtain a nucleic acid sequence corresponding to each sequence of said clique: (B) selecting first and second of the nucleic acid sequences of step (A); (C) aligning the first and second sequences so as to maximize the number of paired matching nucleotides; (D) determining the number of matching nucleotides; (E) arranging the first and second sequences of step (B) in a matrix, wherein: (F)(i) if the number of pairs of matching nucleotides is less than or equal to a predetermined threshold, then the first and second sequences are associated with each other in the matrix; and (F)(ii) if the number of pairs of matching nucleotides is greater than the threshold, then the first and second sequences are non-associated with each other in the matrix; and (G) repeating steps (B) to (F) for a different pair of first and second sequences so as to form one or more cliques, each clique comprising a set of sequences wherein each sequence is associated with every other sequence.

50. The method of claim 49 wherein each block sequence is six blocks in length, and each x-mer is a 4-mer.

51. A method of processing block sequences, the method comprising;
(I) providing a database comprising a plurality of specific block sequences six blocks in length;
(II) determining which of the plurality of block sequences meet the conditions assigned in step (c) of claim 29 for a predetermined threshold for a first toplogical sequence six blocks in length;
(III) storing the specific block sequences determined in step (II) to meet the assigned conditions into a database;
(IV) repeating steps (II) and (III) for a second topological sequence six blocks in length;
(V) determining whether each specific block sequence stored in step (III) meet conditions assigned according to step (iii) of claim 32 wherein the first and second topological block sequences of step (iii) correspond to the first and second toplogical sequences of steps (II) and (IV);
(VI) storing the specific block sequences determined in step (V) to meet the assigned conditions into a database;
(VII) selecting first and second sequences from the database of step (VI);
(VIII) aligning the first and second sequences of step (VII) so as to maximize the number of paired blocks having the same designation;
(IX) determining the number of matching pairs of blocks of step (VIII);
(X) storing matched pair blocks onto a computer readable medium in association with each other, as in a matrix, wherein: (X)(i) if the number of paired blocks having the same designation is less than or equal to the threshold, then the first and second sequences are associated with each other, and
(XI) repeating steps (VIII) to (X) for a different pair of first and second sequences so as to form one or more cliques, each clique comprising a set of sequences wherein each sequence is associated with every other sequence.

52. A method of processing a family of topological block sequences useful in creating a family of nucleic acid molecules, the method comprising:
(a) providing first and second topological block sequences, each sequence having a predetermined number of core blocks and a predetermined number of variable blocks;
(b) aligning the first and second sequences with each other such that at least one block of the first sequence is paired with at least one block of the second sequence in an aligned arrangement;
(c) assigning conditions to the variable blocks of the first and second sequences, as necessary, such that the sum of (i) the number of pairs of aligned core blocks, and (ii) the number of pairs of aligned variable blocks, in which both variable blocks are permitted to have the same designation, does not exceed a predetermined threshold;
(d) storing the conditions determined for each variable block of the first and second sequences in a computer readable medium in association with the respective first and second sequences;
(e) optionally, repeating steps (b) through (d) for a different said aligned arrangement of step (b); and
(f) optionally, repeating steps (b) through (e) for a different pair of first and second topological block sequences.

53. The method of claim 52, further comprising:
(h) providing a database of specific block sequences, each block of each sequence having a specific designation associated therewith;
(i) determining which of the plurality of specific block sequences meet the conditions assigned in step (c);
(j) storing the specific block sequences determined in step (i) to meet the conditions assigned in step (c) into a database.

54. A method of providing a family of nucleotide sequences, comprising assigning an x-mer to each specific designation of a block sequence of step (j) of claim 53.

55. The method of claim 52, 53 or 54, wherein the first and second sequences of step (b) have the same topology as each other.

56. The method of claim 52, 53, or 54, wherein the first and second sequences of step (b) have a different topology from each other.

57. The method of any of claims 52 to 56, wherein each topological block sequence has at least 5 blocks.

58. The method of any of claims 52 to 57, wherein each topological block sequence consists of 6 blocks, 7 blocks, or 8 blocks.

59. The method of any of claims 52 to 58, wherein each topological block sequence consists of 6 blocks.

60. The method of any of claims 57 to 59, wherein the number of core blocks exceeds the number of variable blocks.

61. The method of claim 59, wherein the number of core blocks is 4 and the number of variable blocks is 2.

62. The method of any of claims 52 to 61 wherein at least one variable block is a terminal block of each topological block sequence.

63. The method of claim 53, wherein:
each topological sequence has 4 core blocks 2 variable blocks;
the first and second sequences of step (b) have the same topology as each other; and
at least one variable block of each topological block sequence is a terminal block.

64. A method of processing a family of topological block sequences useful in creating a family of nucleic acid molecules, the method comprising:
(a) providing a first pair of first and second topological block sequences, each sequence having c core blocks and v variable blocks, c and v being natural numbers, the first and second topological sequences having the same topology as each other;
(b) aligning the sequences with each other such that the core blocks of each sequence are paired with each other and the variable blocks of each sequence are paired with each other in an aligned arrangement;
(c) assigning conditions to the variable blocks of the first and second sequences that are necessary to preclude the sum of (i) the number of pairs of aligned core blocks, and (ii) the number of pairs of aligned variable blocks, in which both variable blocks are permitted to have the same designation, from exceeding a predetermined threshold; and
(d) storing the necessary conditions determined for each variable block of the first and second sequences in a computer readable medium in association with the respective first and second sequences.

65. The method of claim 64, further comprising, (e) providing a second pair of said first and second topological block sequences, each sequence having c core blocks and v variable blocks, wherein the topology of the second pair of sequences is different from the topology of the first pair of sequences, and conducting steps (b) to (d) for the second pair of sequences.

66. The method of claim 65, further comprising, (f) providing a database of specific block sequences, each block of each sequence having a specific designation associated therewith, (g) determining which of the plurality of specific block sequences meet the conditions stored in step (d) in association with the first pair of topological sequences; (h) repeating step (g) for the conditions stored in step (d) in association with the second pair of topological sequences; and (i) storing the specific block sequences determined in steps (g) and (h) into a database.

67. The method of claim 66, further comprising the steps of (j) selecting a first sequence from the database of step (i); (k) selecting a second sequence from the database of step (i); (l) aligning the first and second sequences so as to maximize the number of paired blocks having the same designation; (m) determining the number of matching pairs; (n) arranging the first and second sequences of step (l) in a matrix, wherein: (n)(i) if the number of paired blocks having the same designation is less than or equal to the threshold of step (c), then the first and second sequences are associated with each other in the matrix; and (n)(ii) if the number of paired blocks having the same designation is greater than the threshold of step (c), then the first and second sequences are non-associated with each other in the matrix; and (o) repeating steps (j) to (n) for a different pair of first and second sequences of step (i).

68. A method of processing a family of topological block sequences useful in creating a family of nucleic acid molecules, the method comprising:
(a) providing a family of topological block sequences, each sequence of the family having a predetermined first number of core blocks and a predetermined second number of variable blocks;
(b) selecting first and second sequences of the family;
(c) aligning the first and second sequences with each other such that at least one block of the first sequence is paired with at least one block of the second sequence in an aligned arrangement;
(d) determining conditions assignable to the variable blocks of the first and second sequences, as necessary, to maintain the condition that the sum of (i) the number of pairs of aligned core blocks, and (ii) the number of pairs of aligned variable blocks, in which both variable blocks are permitted to have the same designation, does not exceed a predetermined threshold; and
(e) storing the conditions determined for each variable block of the first and second sequences in a computer readable medium in association with the respective first and second sequences;
(f) optionally, repeating steps (c) through (e) for a different arrangement of step (c); and
(g) optionally, repeating steps (b) through (f) for different first and second toplogical sequences.
